(19) United States
(12) Patent Application Publication
(10) Pub. No.: US 2010/0303808 A1
(43) Pub. Date: Dec. 2, 2010

(54) HUMANIZED ANTI-CD20 ANTIBODIES AND METHODS OF USE

(76) Inventor: David G. Williams, Epsom (GB)

Correspondence Address: BRINKS, HOFER, GILSON & LIONE, P.O. BOX 110285, RESEARCH TRIANGLE PARK, NC, 27709, US

(21) Appl. No.: 12/720,483

(22) Filed: Mar. 9, 2010

Related U.S. Application Data
(63) Continuation of application No. PCT/US2010/000449, filed on Feb. 16, 2010.
(60) Provisional application No. 61/152,778, filed on Feb. 16, 2009.
(60) Provisional application No. 61/153,499, filed on Feb. 18, 2009.

(51) Int. Cl. A61K 39/395; C07K 16/28; C07H 21/04; C12N 1/21; A61P 37/00

(52) U.S. Cl. 424/133.1; 530/387.3; 536/23.53; 435/252.3

(57) ABSTRACT

Humanized anti-CD20 antibodies are provided that may be used for the treatment of diseases and conditions associated with CD20-expressing cells. Also provided are nucleic acids enoding such antibodies, methods of making such antibodies, and compositions comprising such antibodies.

### CROSS-REFERENCE TO RELATED APPLICATIONS

This application is a continuation of International Application No. PCT/US2010/000449, filed Feb. 16, 2010; this application also claims the benefit of U.S. Provisional Application Nos. 61/152,778 and 61/153,499, filed Feb. 16, 2009 and Feb. 18, 2009, respectively; the entire content of all of these applications is hereby incorporated by reference herein.

### REFERENCE TO SEQUENCE LISTING SUBMITTED VIA EFS-WEB

This application is being filed electronically via EFS-Web and includes an electronically submitted sequence listing in .txt format. The .txt file contains a sequence listing entitled “13790-115.txt” created on Mar. 9, 2010 and is 92,095 bytes in size. The sequence listing contained in this .txt file is part of the specification and is hereby incorporated by reference herein in its entirety.

### FIELD OF THE INVENTION

The invention relates to humanized anti-CD20 antibodies and their use in the treatment of various diseases and conditions. The invention also relates to nucleic acids encoding such antibodies, methods of making such antibodies, and compositions comprising such antibodies.

### BACKGROUND

Human CD20 (also called human B-lymphocyte-restricted differentiation antigen as well as Bp35) is a hydrophobic transmembrane protein with a molecular weight of approximately 35 kilodaltons located on pre-B and mature B lymphocytes. CD20 is not found on hematopoietic stem cells, pro-B cells, normal plasma cells or other normal tissues.

Human CD20 is a good target for immunotherapy of B cell neoplasms because it is expressed on the surface of over 90% of malignant B cells but not expressed on hemopoietic stem cells, normal plasma cells, myeloid, T lineage, endothelial, or other nonlymphoid cells. Human CD20 is also a good target for immunotherapy of autoimmune diseases, as B-cell depletion has been shown to be an effective strategy for treating autoimmune diseases. For example, rituximab, the chimeric anti-CD20 antibody C2B8 (available commercially as RITUXAN® and marketed by Biogen Idec Inc. and Genentech, Inc.), is approved for various indications, including non-Hodgkin's lymphoma and rheumatoid arthritis.

Upon binding antibody, CD20 does not significantly modulate nor is it shed. A plethora of antibody effector functions have been shown to be recruited by anti-CD20 antibodies, including antibody-dependent cell-mediated cytotoxicity (ADCC) by mononuclear effector cells, complement-dependent lysis, initiation of intracellular signals such as calcium fluxes, inhibition of cell growth, and induction of cell differentiation. Anti-CD20 antibodies have also been shown to induce apoptosis of malignant B cell lines, especially after intensive cross-linking, for example, by cells expressing receptors for the Fc domain of IgG (FcγR).

The anti-CD20 monoclonal antibodies rituximab, anti-B1, and 1F5 have similar apoptotic effects on B cell lines.

Limitations of current murine and chimeric antibodies include the human anti-mouse antibody (HAMA) response and the human anti-chimeric antibody (HACA) response.

### BRIEF SUMMARY

In one aspect, a humanized antibody is provided that comprises:

(a) a light chain comprising a light chain variable region comprising an amino acid sequence selected from the group consisting of:

(SEQ ID NO: 1)

(1) Glu Ile Val Leu Thr Gln Ser Pro Ser Ser Leu
Ser Ala Ser Val Gly Asp Arg Val Thr Ile Thr Cys
Arg Ala Ser Ser Ser Leu Ser Phe Met His Trp Tyr
Gln Gln Lys Pro Gly Lys Ala Pro Lys Xaa₄₅
Xaa₄₆ Ile Tyr Ala Thr Ser Asn Leu Ala Ser Gly Val
Pro Ser Arg Phe Ser Gly Ser Gly Ser Gly Thr Asp
Xaa₇₀ Thr Leu Thr Ile Ser Ser Leu Gln Pro Glu Asp
Phe Ala Thr Tyr Xaa₈₆ Cys His Gln Trp Ser Ser Asn
Pro Leu Thr Phe Gly Gly Gly Thr Lys Val Glu Ile
Lys

wherein
   Xaa₄₅ is Pro or Leu;
   Xaa₄₆ is Trp or Leu;
   Xaa₇₀ is Tyr or Phe; and
   Xaa₈₆ is Tyr or Phe; and

(SEQ ID NO: 2)

(2) Glu Ile Val Leu Thr Gln Ser Pro Gly Thr Leu
Ser Leu Ser Pro Gly Glu Arg Ala Thr Leu Ser Cys
Arg Ala Ser Ser Ser Leu Ser Phe Met His Trp Tyr
Gln Gln Lys Pro Gly Gln Ala Pro Arg Xaa₄₅ Xaa₄₆
Ile Tyr Ala Thr Ser Asn Leu Ala Ser Gly Ile Pro
Ala Arg Phe Ser Gly Ser Gly Ser Gly Thr Asp Xaa₇₀
Thr Leu Thr Ile Ser Arg Leu Glu Pro Glu Asp Phe
Ala Val Tyr Phe Cys His Gln Trp Ser Ser Asn
Pro Leu Thr Phe Gly Gln Gly Thr Lys Val Glu Leu
Lys

wherein
   Xaa₄₅ is Pro or Leu;
   Xaa₄₆ is Trp or Leu; and
   Xaa₇₀ is Tyr or Phe; and

(b) a heavy chain comprising a heavy chain variable region comprising the following amino acid sequence:

(SEQ ID NO: 3)

Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr
Xaa₇₀ Thr Xaa₇₂ Asp Xaa₇₄ Ser Ala Asn Thr Ala
Tyr Met Glu Leu Ser Ser Leu Lys Ser Glu Asp
Thr Ala Val Tyr Tyr Cys Ala Arg Ser His
Tyr Gly Ser Asn Tyr Val Asp Tyr Phe Asp Tyr
Trp Gly Gln Gly Thr Leu Val Thr Val Ser Ser

wherein
Xaa₆₈ is Val or Ala;
Xaa₇₀ is Val or Leu;
Xaa₇₂ is Arg or Ala; and
Xaa₇₄ is Thr or Lys; and
   wherein the humanized antibody binds to human CD-20.

In another aspect, a humanized antibody is provided that comprises a light chain comprising a light chain variable region comprising the following amino acid sequence:

(SEQ ID NO: 1)

Glu Ile Val Leu Thr Gln Ser Pro Ser Ser Leu Ser
Ala Ser Val Gly Asp Arg Val Thr Ile Thr Cys Arg
Ala Ser Ser Ser Leu Ser Phe Met His Trp Tyr Gln
Gln Lys Pro Gly Lys Ala Pro Lys Xaa₄₅ Xaa₄₆ Ile
Tyr Ala Thr Ser Asn Leu Ala Ser Gly Val Pro Ser
Arg Phe Ser Gly Ser Gly Ser Gly Thr Asp Xaa₇₀ Thr
Leu Thr Ile Ser Ser Leu Gln Pro Glu Asp Phe Ala
Thr Tyr Xaa₈₆ Cys His Gln Trp Ser Ser Asn
Pro Leu Thr Phe Gly Gly Gly Thr Lys Val Glu Ile
Lys

wherein
   Xaa₄₅ is Pro or Leu;
   Xaa₄₆ is Trp or Leu;
   Xaa₇₀ is Tyr or Phe; and
   Xaa₈₆ is Tyr or Phe;
      wherein the humanized antibody binds to human CD-20.

In yet another aspect, a humanized antibody is provided that comprises a light chain comprising a light chain variable region comprising the following amino acid sequence:

(SEQ ID NO: 2)

Glu Ile Val Leu Thr Gln Ser Pro Gly Thr Leu Ser
Leu Ser Pro Gly Glu Arg Ala Thr Leu Ser Cys Arg
Ala Ser Ser Ser Leu Ser Phe Met His Trp Tyr Gln
Gln Lys Pro Gly Gln Ala Pro Arg Xaa₄₅ Xaa₄₆ Ile
Tyr Ala Thr Ser Asn Leu Ala Ser Gly Ile Pro Ala
Arg Phe Ser Gly Ser Gly Ser Gly Thr Asp Xaa₇₀ Thr
Leu Thr Ile Ser Arg Leu Glu Pro Glu Asp Phe Ala
Val Tyr Phe Cys His Gln Trp Ser Ser Asn Pro Leu
Thr Phe Gly Gln Gly Thr Lys Val Glu Leu Lys

wherein
   Xaa₄₅ is Pro or Leu;
   Xaa₄₆ is Trp or Leu; and
   Xaa₇₀ is Tyr or Phe;
      wherein the humanized antibody binds to human CD-20.

In a further aspect, a humanized antibody is provided that comprises a heavy chain comprising a heavy chain variable region comprising the following amino acid sequence:

(SEQ ID NO: 3)

Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr Xaa₇₀ Thr
Xaa₇₂ Asp Xaa₇₄ Ser Ala Asn Thr Ala Tyr Met Glu
Leu Ser Ser Leu Lys Ser Glu Asp Thr Ala Val Tyr
Tyr Cys Ala Arg Ser His Tyr Gly Ser Asn Tyr Val
Asp Tyr Phe Asp Tyr Trp Gly Gln Gly Thr Leu Val
Thr Val Ser Ser

wherein
   Xaa₆₈ is Val or Ala;
   Xaa₇₀ is Val or Leu;
   Xaa₇₂ is Arg or Ala; and
   Xaa₇₄ is Thr or Lys;
      wherein the humanized antibody is capable of binding to human CD-20.

In yet a further aspect, a method of treating a B-cell disorder in a subject is provided. The method comprises administering to a subject in need thereof a therapeutically effective amount of any of the humanized antibodies disclosed herein.

In another aspect, a method of preventing a B-cell disorder in a subject is provided comprising administering to the subject a prophylactically effective amount of any of the humanized antibodies disclosed herein.

In a further aspect, a humanized anti-CD20 antibody composition comprising any of the humanized antibodies disclosed herein, wherein at least 90% of the N-glycans present in the composition are GlcNAc₂Man₃GlcNAc₂ (G0).

In another aspect, a pharmaceutical composition is provided comprising any of the humanized antibodies disclosed herein and a pharmaceutically acceptable excipient.

In yet a further aspect, an isolated nucleic acid is provided comprising a nucleic acid sequence encoding any of the humanized antibodies disclosed herein.

Also provided are host cells comprising any of the aforementioned isolated nucleic acids.

### BRIEF DESCRIPTION OF THE DRAWINGS

FIG. 1A provides one embodiment of the nucleic acid sequences of the various nucleic acid molecules (including the X62109 signal peptide, U00570 Framework (FR) sequences, and 1F5VH complementarity determining regions (CDRs)) used to generate the 1F5RHAss nucleic acid molecule. FIG. 1B illustrates the CDRs 1, 2, and 3 from 1F5VH (SEQ ID NOS: 77-79, respectively, from left to right) that were grafted into the acceptor FR of U00570 (SEQ ID NOS: 80-83, respectively, from left to right) to generate 1F5RHA.

FIG. 2 provides the nucleic acid (SEQ ID NO: 15) and amino acid sequence (SEQ ID NO: 16) of the optimized 1F5RHA nucleic acid construct, containing no mutations in the framework region (FR).

FIG. 3 provides the nucleic acid (SEQ ID NO: 17) and amino acid sequence (SEQ ID NO: 18) of the optimized 1F5RHB nucleic acid construct containing back-mutations at the 4 non-conserved vernier residues as follows: V67A; V69L; R71A; and T73K (Kabat numbering).

FIG. 4A provides sequences of framework regions (FRs) and complementarity determining regions (CDRs) used to generate one example of a humanized anti-CD20 antibody, 1F5RKA11ss. FIG. 4B illustrates the amino acid sequences of the FR (SEQ ID NOS: 87-90, respectively, from left to right) and CDRs (SEQ ID NOS: 84-86, respectively, from left to right) used to generate 1F5RKA11.

FIG. 5 provides the nucleic acid sequence (SEQ ID NO: 30) and amino acid sequence (SEQ ID NO: 31) of an example of an optimized antibody based on 1F5RKA11ss to generate the 1F5RKA11 construct. This construct contains no FR mutations.

FIG. 6 provides the nucleic acid (SEQ ID NO: 32) and amino acid sequence (SEQ ID NO: 33) of an example of a humanized antibody, 1F5RKB11, based on 1F5RKA11, containing back-mutations at the 3 non-conserved vernier residues as follows: L46P; L47W; and F71Y (Kabat numbering); and at VH/VK interface residue Y87F (Kabat numbering).

FIG. 7A provides sequences of framework regions (FRs) and complementarity determining regions (CDRs) used to generate one example of a humanized anti-CD20 antibody, 1F5RKA12ss. FIG. 7B illustrates the CDRs 1, 2, and 3 from 1F5VK (SEQ ID NOS: 91-93, respectively, from left to right) that were grafted into the acceptor FR of human AY263415 (SEQ ID NOS: 94-97, respectively, from left to right) to generate 1F5RKA12.

FIG. 8 provides the nucleic acid sequence (SEQ ID NO: 44) and amino acid sequence (SEQ ID NO: 45) of an example of an optimized antibody based on 1F5RKA12ss to generate the 1F5RKA12 construct. This construct contains no FR mutations.

FIG. 9 provides the nucleic acid sequence (SEQ ID NO: 46) and amino acid sequence (SEQ ID NO: 47) of an example of a humanized anti-CD20 antibody, 1F5RKB12, based on 1F5RKA12, containing back-mutations at the 3 non-conserved vernier residues as follows: L46P; L47W; and F71Y (Kabat numbering).

FIG. 10A provides an amino acid sequence alignment of examples of variants of 1F5RHA. FIG. 10B provides an amino acid sequence alignment of examples of variants of 1F5RKA11. FIG. 10C provides an amino acid sequence alignment of examples of variants of 1F5RKA12. The CDR amino acid residues are indicated by boxes labeled “CDR1”, “CDR2”, and “CDR3”; the underlined amino acids indicate mouse FW residues that were introduced into the humanized sequences.

FIGS. 11A-E provide amino acid sequences of the mature variable and constant regions from 1F5RKG11, 1F5RKB11, 1F5RKF12, 1F5RKB12, and 1F5RHA proteins. FIG. 11A provides the amino acid sequence for the mature variable and constant (underlined) regions of 1F5RKG11 (SEQ ID NO: 60). FIG. 11B provides the amino acid sequence for the mature variable and constant (underlined) regions of 1F5RKB11 (SEQ ID NO: 61). FIG. 11C provides the amino acid sequence for the mature variable and constant (underlined) regions of 1F5RKF12 (SEQ ID NO: 62). FIG. 11D provides the amino acid sequence for the mature variable and constant (underlined) regions of 1F5RKB12 (SEQ ID NO: 63). FIG. 11E provides the amino acid sequence for the mature variable and constant (underlined) regions of 1F5RHA (SEQ ID NO: 64). The constant regions of the amino acid sequences are underlined.

FIG. 12A provides the results of Raji binding assays for chimeric VK×1F5RHA (RHA) and chimeric VH×(1F5RKA11, 1F5RKA12, or 1F5RKB11) compared with chimeric c1F5 antibody (Chimeric). FIG. 12B provides the results of Raji binding assays for humanized 1F5RHA (RHA) or 1F5RHB (RHB) in association with chimeric VK, compared with Rituxan. FIGS. 12C and 12D provide results of Raji binding assays for humanized 1F5VK versions (KB11, etc.) in association with chimeric VH, compared to the chimeric antibody c1F5.

FIGS. 13A and 13B provide examples of Raji cell binding of antibodies encoded by humanized 1F5RHA in association with the humanized 1F5 kappa chains indicated, compared with the chimeric version of 1F5. Measurements are averages of duplicate wells.

FIG. 14 provides an example Raji cell binding of antibodies encoded by humanized 1F5RHA in association with the humanized 1F5 kappa chains indicated, compared with the chimeric version of 1F5. Measurements are averages of quadruplicate wells. Error bars indicate the standard deviation between the quadruplicates.

FIG. 15 provides an example of thermostability results for some embodiments of humanized anti-CD20 antibodies. Each antibody was diluted to 1 μg/ml in medium/PBS, heated for 10 minutes at the indicated temperature, then cooled to 4° C. before Raji cell binding ELISA at room temperature. The humanized antibodies (apart from Rituxan) are encoded by 1F5RHA together with the indicated light chain construct.

FIG. 16 provides an example of the mean fluorescence intensity in a fluorescent antibody binding assay on Raji cells for several embodiments of humanized anti-CD20 antibodies.

FIG. 17 provides an example of a pre-optimized chimeric c1F5VK nucleic acid (SEQ ID NO: 65) and corresponding amino acid sequence (SEQ ID NO: 66).

FIG. 18 provides an example of an optimized chimeric c1F5VK nucleic acid (SEQ ID NO: 67) and corresponding amino acid sequence (SEQ ID NO: 68).

FIG. 19 provides an example of a pre-optimized chimeric c1F5VH nucleic acid (SEQ ID NO: 69) and corresponding amino acid sequence (SEQ ID NO: 70).

FIG. 20 provides an example of an optimized chimeric c1F5VH nucleic acid (SEQ ID NO: 71) and corresponding amino acid sequence (SEQ ID NO: 72).

FIG. 21 shows the G0 glycan structure.

FIG. 22 provides an example of a chimeric c1F5VH protein sequence (mature variable and constant region) (SEQ ID NO: 73). The constant region of the amino acid sequence is underlined.

FIG. 23 provides an example of a chimeric c1F5VK protein sequence (mature variable and constant region) (SEQ ID NO: 74). The constant region of the amino acid sequence is underlined.

FIG. 24 shows the results of the CDC assay of various concentrations of the tested anti-CD20 antibodies as explained in Example 5.

FIG. 25 shows the results of an assay measuring the dissociation of anti-CD20 antibodies (i.e., off-rate) from Raji cells as explained in Example 5.

FIG. 26 shows a bar graph of the half-lives obtained from one of the off-rate studies on anti-CD20 antibodies explained in Example 5.

FIG. 27 shows the results of a B-cell depletion assay using anti-CD20 antibodies at various concentrations.

FIG. 28 shows the results of a B-cell depletion assay using anti-CD20 antibodies at various concentrations in the presence of anti-CD16 antibody.

### DETAILED DESCRIPTION

The present invention relates to humanized 1F5 antibodies that bind to, or are capable of binding to, CD20, as well as to compositions comprising those antibodies. The present invention also relates to nucleic acid molecules encoding humanized 1F5 antibodies. That is, humanized anti-CD20 antibodies and nucleic acid molecules encoding such anti-CD20 antibodies are provided.

The antibodies and compositions thereof provided herein are useful in methods for treating diseases and conditions associated with cells expressing CD20 (such as B cells), including lymphomas, autoimmune diseases, and transplant rejections. The compositions include the humanized anti-CD20 antibodies, antigen-binding fragments of the humanized anti-CD20 antibodies, and pharmaceutical compositions of such antibodies. The compositions also include isolated nucleic acid molecules encoding the humanized anti-CD20 antibodies discussed herein, vectors comprising the nucleic acid molecules that encode the humanized anti-CD20 antibodies, host cells (including transfectomas and hybridomas) transformed with the vectors or incorporating the nucleic acid molecules that express the humanized antibodies, pharmaceutical formulations of the anti-CD20 humanized antibodies, and methods of making and using the same. Methods for treating or preventing diseases or disorders associated with cells expressing CD20 (such as B-cell disorders) are also provided.

Prior to describing the invention in further detail, the following terms will first be defined. Unless otherwise noted, all nucleic acids sequences are written from left to right in 5′ to 3′ orientation, and all amino acid sequences are written from left to right in amino-terminal to carboxy-terminal orientation.

### DEFINITIONS

The terms “antibody” and “immunoglobulin” are used interchangeably and are used in their broadest sense herein. Specifically, the term includes monoclonal antibodies, multispecific antibodies, antibody fragments, and other antibodies and immunoglobulins so long as they exhibit the desired biological activity and function.

“Antibody fragments” and “antibody fragment” comprise a portion of a full length antibody, and generally include the variable region thereof.

As used herein, a “CD20” or “human CD20” (also called human B-lymphocyte-restricted differentiation antigen, Bp35) refers to a transmembrane phosphoprotein with a molecular weight of approximately 35 kilodaltons that is expressed on normal and malignant B cells.

An “anti-CD20 antibody” as used herein means an antibody that specifically binds human CD20.

As used herein, a “chimeric antibody” refers to any antibody in which the immunoreactive or binding region or site is obtained or derived from a first species and the constant region (which may be intact, partial or modified) is obtained from a second species. For example, the target binding region or site is derived from a non-human source (e.g., mouse or primate) and the constant region is derived from a human antibody.

A “humanized antibody” refers to a polypeptide comprising at least a portion of a modified variable region of a human antibody wherein a portion of the variable region, preferably a portion substantially less than the intact human variable domain, has been substituted by the corresponding sequence from a non-human species and wherein the modified variable region is linked to at least another part of another protein, preferably the constant region of a human antibody. The expression “humanized antibodies” includes human antibodies in which one or more complementarity determining region (“CDR”) amino acid residues and/or one or more framework region (“FW” or “FR”) amino acid residues are substituted by amino acid residues from analogous sites in rodent or other non-human antibodies that are capable of binding to CD20. The expression “humanized antibody” also includes an immunoglobulin amino acid sequence variant or fragment thereof that is capable of binding to CD20 and that comprises an FR having substantially the amino acid sequence of a human immunoglobulin and a CDR having substantially the amino acid sequence of a non-human immunoglobulin.

Immunoglobulin heavy chains can be classified as gamma (γ), mu (μ), alpha (α), delta (δ), or epsilon (ε), with some subclasses among them (e.g., γ1-γ4). The classification (or “class”) of the antibody as IgG, IgM, IgA, IgD, or IgE, respectively, is determined by the nature of the heavy chain which confers functional specialization to the immunoglobulin. Immunoglobulin can be classified into subclasses (or “isotypes”) for example, IgG₁, IgG₂, IgG₃, IgG₄, IgA₁, etc. Such isotypes are well-characterized and confers additional functional specialization to the immunoglobulin. With regard to IgG, a standard immunoglobulin molecule comprises two identical light chain polypeptides and two identical heavy chain polypeptides. The four chains are typically joined by disulfide bonds in a “Y” configuration where the light chains bracket the heavy chains starting at the mouth of the “Y” and continuing through the variable region.

There are two types of light chains: kappa (κ) or lambda (λ). Each heavy chain class may be bound with either a kappa or lambda light chain. In general, the light and heavy chains are covalently bonded to each other, and the “tail” portions of the two heavy chains are bonded to each other by covalent disulfide linkages and non-covalent linkages when the immunoglobulins are generated by either B cell hybridomas, B cells, or genetically engineered host cells. In the heavy chain, the amino acid sequences run from the amino-terminus at the forked ends of the Y configuration to the carboxyl-terminus at the bottom of each chain.

Both the light and heavy chains are divided into regions of structural and functional homology referred to as “constant regions” and “variable regions.” The terms “constant” and “variable” are used functionally. The variable domains of both the light (V_{L} or VL) and heavy (V_{H} or VH) chain portions determine antigen recognition and specificity. Conversely, the constant domains of the light chain (C_{L}) and the heavy chain (C_{H1}, C_{H2}, or C_{H3}) confer important biological properties such as secretion, transplacental mobility, Fc receptor binding, complement binding, and the like. By convention the numbering of the constant region domains increases as they become more distal from the antigen binding site or amino-terminus of the antibody. The variable region is at the amino-terminus and the constant region is at the carboxyl-terminus; the C_{H3} and C_{L} domains actually comprise the carboxy-terminus of the heavy and light chain, respectively.

The expressions “complementarity determining region,” “hypervariable region,” and “CDR” refer to one or more of the hyper-variable or complementarity determining regions (CDRs) found in the variable regions of light or heavy chains of an antibody (See, Kabat, E. A. et al., Sequences of Proteins of Immunological Interest, National Institutes of Health, Bethesda, Md., (1987)). These expressions include the hypervariable regions as defined by Kabat et al. (“Sequences of Proteins of Immunological Interest,” Kabat E., et al., US Dept. of Health and Human Services, 1983) or the hypervariable loops in 3-dimensional structures of antibodies (Chothia and Lesk, J Mol. Biol. 196 901-917 (1987)). The CDRs in each chain are held in close proximity by framework regions and, together with the CDRs from the other chain, contribute to the formation of the antigen binding site.

The expressions “framework region” and “FR” refer to one or more of the framework regions within the variable regions of the light and heavy chains of an antibody (See, Kabat, E. A. et al., Sequences of Proteins of Immunological Interest, National Institutes of Health, Bethesda, Md., (1987)). These expressions include those amino acid sequences of both light and heavy chains of an antibody, situated between the amino terminus and the first CDR, those interposed between the CDRs, and those between the third CDR and the start of the constant region.

CDR and FR residues can be determined according to a standard sequence definition (Kabat et al., Sequences of Proteins of Immunological Interest, National Institutes of Health, Bethesda Md. (1987)), and a structural definition (as in Chothia and Lesk, J. Mot. Biol. 196:901-217 (1987)).

Where indicated herein, reference is made to the numbering scheme from Kabat, E. A., et al., Sequences of Proteins of Immunological Interest (National Institutes of Health, Bethesda, Md. (1987) and (1991). Kabat uses a method for assigning a residue number to each amino acid in a listed sequence, and this method for assigning residue numbers has become standard in the field. Where indicated, the Kabat numbering scheme is followed in this description. Where Kabat numbering is not indicated, sequential amino acid sequence numbering is used (i.e., the amino acids in a sequence are numbered using sequential integers (1, 2, 3, etc.) from left to right in amino-terminal to carboxy-terminal orientation).

An “antigen-binding fragment” of an antibody refers to biologically active fragments of the antibodies disclosed herein that function essentially the same as a full-length 1F5 antibody to bind to CD20. Such fragments comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

“Fv” is the minimum antibody fragment which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

The terms “rituximab” or “RITUXAN®” refer to a genetically engineered chimeric murine/human monoclonal antibody directed against CD20 that has the amino acid-sequence of the antibody designated “C2B8” in U.S. Pat. No. 5,736,137.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII.

“Affinity” of an antibody for an antigen or epitope is a term well understood in the art and means the extent, or strength, of binding of an antibody to an epitope. Affinity may be measured and/or expressed in a number of ways known in the art, including, but not limited to, equilibrium dissociation constant (K_{D} or K_{d}, which can be defined as the ratio of the off-rate and on-rate of the antibody, i.e., K_{off}/Kₒₙ), apparent equilibrium dissociation constant (K_{D′} or K_{d′}), and IC50 (amount needed to effect 50% inhibition in a competition assay); relative affinity of humanized antibodies may also be determined as compared to, for example, related murine or chimeric antibodies. It is understood that, for purposes of this invention, an affinity is an average affinity for a given population of antibodies which bind to an antigen or epitope. Affinity (or relative affinity) for the humanized anti-CD20 antibodies described herein may be measured using an enzyme-linked immunosorbent assay (ELISA) or a fluorescent-activated cell sorting (FACS) assay as described in the Examples herein.

“Off-rate” means the dissociation rate constant (K_{off}) of an antibody from an antibody/antigen complex. Thus, antibodies with lower off-rates remain bound to the antibody longer than antibodies with higher off-rates.

“On-rate” means the association rate constant (Kₒₙ) of an antibody to an antigen to form an antibody/antigen complex.

An “effective amount” refers to an amount effective, at dosages and for periods of time necessary, to achieve the desired therapeutic or prophylactic result.

A “therapeutically effective amount” of an antibody may vary according to factors such as the disease state, age, sex, and weight of the individual, and the ability of the antibody to elicit a desired response in the individual. A therapeutically effective amount is also one in which any toxic or detrimental effects of the antibody are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount may be less than the therapeutically effective amount.

“B-cell disorders” includes a variety of disorders, including, but not limited to,

B-cell malignancies, autoimmune disorders, B-cell lymphomas, B-cell leukemias, and other disorders.

An “autoimmune disorder” or “autoimmune disease” as used herein refers to a non-malignant disease or disorder arising from and directed against an individual's own tissues. Examples of autoimmune diseases or disorders include, but are not limited to, inflammatory responses such as inflammatory skin diseases including psoriasis and dermatitis (for example, atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g. Type I diabetes mellitus or insulin dependent diabetes mellitis); multiple sclerosis; Raynaud's syndrome; autoimmune thyroiditis; allergic encephalomyelitis; Sjögren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; bullous pemphigoid; pemphigus; autoimmune polyendocrinopathies; Reiter's disease; stiff-man syndrome; Behcet's disease; giant cell arteritis; immune complex nephritis; IgA nephropathy; IgM polyneuropathies; immune thrombocytopenic purpura (ITP) or autoimmune thrombocytopenia etc.

### Humanized 1F5 Antibodies:

The invention provides humanized 1F5 antibodies and antigen-binding fragments thereof that are capable of binding to human CD20 (i.e., humanized anti-CD20 antibodies).

In one embodiment, a humanized antibody (including antigen-binding fragments thereof) is provided, comprising a light chain comprising a light chain variable region comprising an amino acid sequence selected from the group consisting of:

(SEQ ID NO: 1)

(1) Glu Ile Val Leu Thr Gln Ser Pro Ser Ser Leu
Ser Ala Ser Val Gly Asp Arg Val Thr Ile Thr Cys
Arg Ala Ser Ser Ser Leu Ser Phe Met His Trp Tyr
Gln Gln Lys Pro Gly Lys Ala Pro Lys Xaa₄₅ Xaa₄₆
Ile Tyr Ala Thr Ser Asn Leu Ala Ser Gly Val Pro
Ser Arg Phe Ser Gly Ser Gly Ser Gly Thr Asp Xaa₇₀
Thr Leu Thr Ile Ser Ser Leu Gln Pro Glu Asp Phe
Ala Thr Tyr Xaa₈₆ Cys His Gln Trp Ser Ser
Asn Pro Leu Thr Phe Gly Gly Gly Thr Lys Val Glu
Ile Lys

wherein:
   Xaa₄₅ is Pro or Leu;
   Xaa₄₆ is Trp or Leu;
   Xaa₇₀ is Tyr or Phe; and
   Xaa₈₆ is Tyr or Phe; and

(SEQ ID NO: 2)

(2) Glu Ile Val Leu Thr Gln Ser Pro Gly Thr Leu
Ser Leu Ser Pro Gly Glu Arg Ala Thr Leu Ser Cys
Arg Ala Ser Ser Ser Leu Ser Phe Met His Trp Tyr
Gln Gln Lys Pro Gly Gln Ala Pro Arg Xaa₄₅ Xaa₄₆
Ile Tyr Ala Thr Ser Asn Leu Ala Ser Gly Ile Pro
Ala Arg Phe Ser Gly Ser Gly Ser Gly Thr Asp Xaa₇₀
Thr Leu Thr Ile Ser Arg Leu Glu Pro Glu Asp Phe
Ala Val Tyr Phe Cys His Gln Trp Ser Ser Asn Pro
Leu Thr Phe Gly Gln Gly Thr Lys Val Glu Leu Lys

wherein:
   Xaa₄₅ is Pro or Leu;
   Xaa₄₆ is Trp or Leu; and
   Xaa₇₀ is Tyr or Phe.

In another embodiment, a humanized antibody (including antigen-binding fragments thereof) is provided comprising a heavy chain comprising a heavy chain variable region comprising the following amino acid sequence:

(SEQ ID NO: 3)

Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr Xaa₇₀ Thr
Xaa₇₂ Asp Xaa₇₄ Ser Ala Asn Thr Ala Tyr Met Glu
Leu Ser Ser Leu Lys Ser Glu Asp Thr Ala Val Tyr
Tyr Cys Ala Arg Ser His Tyr Gly Ser Asn Tyr Val
Asp Tyr Phe Asp Tyr Trp Gly Gln Gly Thr Leu Val
Thr Val Ser Ser

wherein:
Xaa₆₈ is Val or Ala;
Xaa₇₀ is Val or Leu;
Xaa₇₂ is Arg or Ala; and
Xaa₇₄ is Thr or Lys.

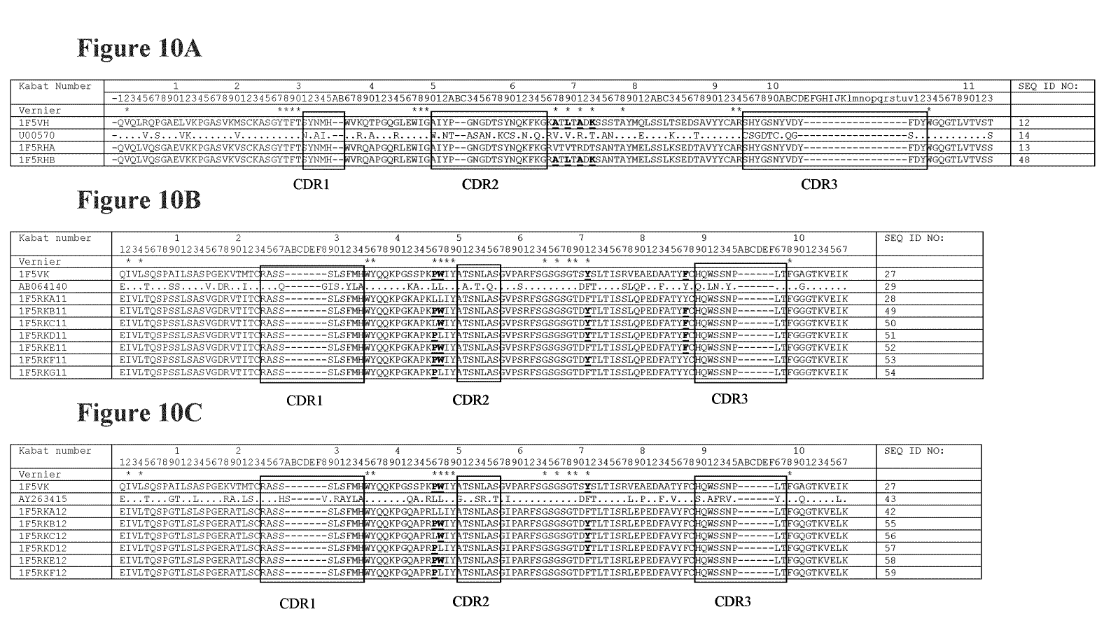

Thus, the light chain variable regions of the anti-CD20 antibodies may have various amino acid sequences, including the following amino acid sequences set forth in FIGS. 10B and 10C: SEQ ID NOS: 28, 49, 50, 51, 52, 53, 54, 42, 55, 56, 57, 58, and 59. In one example, the light chain variable region of the anti-CD20 antibody comprises the amino acid sequence of SEQ ID NO: 49. In another example, the light chain variable region of the anti-CD20 antibody comprises the amino acid sequence of SEQ ID NO: 54. In still another example, the light chain variable region of the anti-CD20 antibody comprises the amino acid sequence of SEQ ID NO: 59. In yet another example, the light chain variable region of the anti-CD20 antibody comprises the amino acid sequence of SEQ ID NO: 55.

The heavy chain variable regions of the anti-CD20 antibodies may have various amino acid sequences, including the following amino acid sequences set forth in FIG. 10A: SEQ ID NO: 13 and SEQ ID NO: 48.

In one embodiment, a humanized antibody (including antigen-binding fragments thereof) is provided comprising

(a) a light chain comprising a light chain variable region comprising an amino acid sequence selected from the group consisting of:

(SEQ ID NO: 1)

(1) Glu Ile Val Leu Thr Gln Ser Pro Ser Ser Leu
Ser Ala Ser Val Gly Asp Arg Val Thr Ile Thr Cys
Arg Ala Ser Ser Ser Leu Ser Phe Met His Trp Tyr
Gln Gln Lys Pro Gly Lys Ala Pro Lys Xaa₄₅ Xaa₄₆
Ile Tyr Ala Thr Ser Asn Leu Ala Ser Gly Val Pro
Ser Arg Phe Ser Gly Ser Gly Ser Gly Thr Asp Xaa₇₀
Thr Leu Thr Ile Ser Ser Leu Gln Pro Glu Asp Phe
Ala Thr Tyr Xaa₈₆ Cys His Gln Trp Ser Ser Asn
Pro Leu Thr Phe Gly Gly Gly Thr Lys Val Glu Ile
Lys

wherein:
   Xaa₄₅ is Pro or Leu;
   Xaa₄₆ is Trp or Leu;
   Xaa₇₀ is Tyr or Phe; and
   Xaa₈₆ is Tyr or Phe; and

(SEQ ID NO: 2)

(2) Glu Ile Val Leu Thr Gln Ser Pro Gly Thr Leu
Ser Leu Ser Pro Gly Glu Arg Ala Thr Leu Ser Cys
Arg Ala Ser Ser Ser Leu Ser Phe Met His Trp Tyr
Gln Gln Lys Pro Gly Gln Ala Pro Arg Xaa₄₅ Xaa₄₆
Ile Tyr Ala Thr Ser Asn Leu Ala Ser Gly Ile Pro
Ala Arg Phe Ser Gly Ser Gly Ser Gly Thr Asp Xaa₇₀
Thr Leu Thr Ile Ser Arg Leu Glu Pro Glu Asp Phe
Ala Val Tyr Phe Cys His Gln Trp Ser Ser Asn Pro
Leu Thr Phe Gly Gln Gly Thr Lys Val Glu Leu Lys

wherein:
   Xaa₄₅ is Pro or Leu;
   Xaa₄₆ is Trp or Leu; and
   Xaa₇₀ is Tyr or Phe; and
      (b) a heavy chain comprising a heavy chain variable region comprising the following amino acid sequence:

(SEQ ID NO: 3)

Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr Xaa₇₀ Thr
Xaa₇₂ Asp Xaa₇₄ Ser Ala Asn Thr Ala Tyr Met Glu
Leu Ser Ser Leu Lys Ser Glu Asp Thr Ala Val Tyr
Tyr Cys Ala Arg Ser His Tyr Gly Ser Asn Tyr Val
Asp Tyr Phe Asp Tyr Trp Gly Gln Gly Thr Leu Val
Thr Val Ser Ser

wherein:
   Xaa₆₈ is Val or Ala;
   Xaa₇₀ is Val or Leu;
   Xaa₇₂ is Arg or Ala; and
   Xaa₇₄ is Thr or Lys.

In one embodiment, the anti-CD20 antibody may comprise (1) a heavy chain comprising a heavy chain variable region having the amino acid sequence set forth in SEQ ID NO: 13 and (2) a light chain comprising a light chain variable region having the amino acid sequence set forth in SEQ ID NO: 49.

In another embodiment, the anti-CD20 antibody may comprise (1) a heavy chain comprising a heavy chain variable region having the amino acid sequence set forth in SEQ ID NO: 13 and (2) a light chain comprising a light chain variable region having the amino acid sequence set forth in SEQ ID NO: 54.

In yet another embodiment, the anti-CD20 antibody may comprise (1) a heavy chain comprising a heavy chain variable region having the amino acid sequence set forth in SEQ ID NO: 13 and (2) a light chain comprising a light chain variable region having the amino acid sequence set forth in SEQ ID NO: 55.

In a further embodiment, the humanized anti-CD20 antibody may comprise (1) a heavy chain comprising a heavy chain variable region having the amino acid sequence set forth in SEQ ID NO: 13 and (2) a light chain comprising a light chain variable region having the amino acid sequence set forth in SEQ ID NO: 59.

The humanized anti-CD20 antibodies herein are capable of binding to human CD20. In some embodiments the humanized anti-CD20 antibodies bind to CD20 with an affinity similar to that of antibody 1F5 (i.e., with an affinity similar to that of the affinity that antibody 1F5 has for human CD20). In other embodiments, the humanized anti-CD20 antibodies bind to CD20 with an affinity at least as great as that of antibody 1F5. In yet another embodiment, the humanized anti-CD20 antibodies bind to CD20 with an affinity greater than that of antibody 1F5. As used herein an “affinity similar to that of antibody 1F5” means an affinity of sufficiently high degree of similarity to the affinity of antibody 1F5 such that one of skill in the art would consider the difference between the affinities to be of little or no biological significance.

In some embodiments the humanized anti-CD20 antibodies bind to CD20 with an affinity similar to that of the chimeric antibody c1F5 described in the Examples herein (i.e., with an affinity similar to that of the affinity that chimeric antibody c1F5 has for human CD20). In other embodiments, the humanized anti-CD20 antibodies bind to CD20 with an affinity at least as great as that of the chimeric antibody c1F5. In yet another embodiment, the humanized anti-CD20 antibodies bind to CD20 with an affinity greater than that of the chimeric antibody c1F5. As used herein an “affinity similar to that of chimeric antibody c1F5” means an affinity of sufficiently high degree of similarity to the affinity of chimeric antibody c1F5 such that one of skill in the art would consider the difference between the affinities to be of little or no biological significance.

In some embodiments the humanized anti-CD20 antibodies bind to CD20 with an off-rate lower than that of chimeric antibody c1F5. In other embodiments, the humanized anti-CD20 antibodies not lower than that of the chimeric antibody c1F5.

In further embodiments, the humanized anti-CD20 antibodies bind to CD20 with an affinity greater than that of rituximab. In other embodiments, the humanized anti-CD20 antibodies bind to CD20 with an off-rate lower than that of rituximab.

The anti-CD20 antibodies may comprise heavy chain constant regions (or portions thereof) and/or light chain constant regions (or portions thereof) of any isotype, allotype, and idiotype. Such heavy and light constant regions may be naturally occurring or may contain deletion, substitution, or addition mutations. Human constant region DNA sequences are known and can be isolated from a variety of human cells. When present on the heavy and/or light chains, the constant regions may be attached to the carboxyl-terminal end of the variable regions of the heavy and/or light chains.

### Antibody Fragments

In some embodiments fragments of the humanized anti-CD20 antibodies described herein are provided, rather than whole antibodies.

Any method available can be used for the production of such antibody fragments. Traditionally, these fragments were derived via proteolytic digestion of intact antibodies. However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from antibody phage libraries. Alternatively, Fab′-SH fragments can be directly recovered from E. coli and chemically coupled to form F(ab′)₂ fragments. According to another approach, F(ab′)₂ fragments can be isolated directly from recombinant host cell culture. In other embodiments, the antibody fragment is a single chain Fv fragment (scFv).

The antibody fragment may be antigen-binding fragments of the humanized anti-CD20 antibodies described herein. Any fragment of a whole antibody may be used so long as the fragment binds to CD20.

### Bispecific Antibodies

The humanized anti-CD20 antibodies may also be used in the production of bispecific antibodies. Bispecific antibodies are antibodies that have binding specificities for at least two different epitopes. Exemplary bispecific antibodies may bind to two different epitopes of the CD20 protein. Other such antibodies may combine a CD20 binding site with a binding site for another protein. Alternatively, an anti-CD20 binding site may be combined with a domain or arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), or NKG2D or other NK cell activating ligand, so as to focus and localize cellular defense mechanisms to the CD20-expressing cell. Bispecific antibodies may also be used to localize cytotoxic agents to cells which express CD20. These antibodies possess a CD20-binding arm and an arm which binds the cytotoxic agent (e.g. saporin, anti-interferon-a, vinca alkaloid, ricin A chain, methotrexate or radioactive isotope hapten). Bispecific antibodies can be prepared as full length antibodies or antibody fragments (e.g. F(ab′)₂ bispecific antibodies).

### Multivalent Antibodies

The humanized anti-CD20 antibodies may also be used in the production of multivalent antibodies. A multivalent antibody may be internalized (and/or catabolized) faster than a bivalent antibody by a cell expressing an antigen to which the antibodies bind. The humanized anti-CD20 antibodies may be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and may comprise two polypeptide chains), wherein the polypeptide chain(s) comprise two or more variable domains.

### Recombinant Production Methods

The humanized anti-CD20 antibodies may be produced by any method available, such as, for example, recombinant expression techniques. Nucleic acids encoding the light and heavy chain variable regions, optionally linked to constant regions, can be inserted into expression vectors. The light and heavy chains can be cloned in the same or different expression vectors. The DNA segments encoding immunoglobulin chains can be operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. In one embodiment, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the antibodies.

The expression vectors may be replicable in any host organism, either as episomes or as an integral part of the host chromosomal DNA. In one embodiment, expression vectors contain selection markers (e.g., ampicillin-resistance, hygromycin-resistance, tetracycline resistance or neomycin resistance) to permit detection of those cells transformed with the desired DNA sequences.

The expression vectors may be used to express the humanized anti-CD20 antibodies from any host cell, including prokaryotic host cells (e.g. E. coli), yeast host cells, mammalian host cells, plant host cells, and insect host cells.

In one embodiment, E. coli is used for production of the humanized antibodies. Other prokaryotic hosts suitable for such use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression of the humanized antibodies. For example, Saccharomyces can be used as a yeast host, with suitable vectors having expression control sequences (e.g., promoters), an origin of replication, termination sequences and the like as desired. Promoters for use in yeast expression techniques include 3-phosphoglycerate kinase and other glycolytic enzymes. Inducible yeast promoters include, among others, promoters from alcohol dehydrogenase, isocytochrome C, and enzymes responsible for maltose and galactose utilization.

In another embodiment, mammalian tissue cell culture may be used to express and produce the humanized anti-CD20 antibodies (e.g., polynucleotides encoding immunoglobulins or fragments thereof). Any mammalian tissue cell may be used in such methods, and a number of suitable host cell lines capable of secreting heterologous proteins (e.g., intact immunoglobulins) have been developed in the art, and include CHO cell lines, various Cos cell lines, HeLa cells, preferably, myeloma cell lines, or transformed B-cells or hybridomas. In one embodiment the cells are nonhuman. Expression vectors for the mammalian cells can include expression control sequences, such as an origin of replication, a promoter, and an enhancer, and necessary processing information sites, such as ribosome binding sites, RNA splice sites, polyadenylation sites, and transcriptional terminator sequences. In one embodiment, the expression control sequences are promoters derived from immunoglobulin genes, SV40, adenovirus, bovine papilloma virus, cytomegalovirus and the like.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. Other methods used to transform mammalian cells include the use of polybrene, protoplast fusion, liposomes, electroporation, and microinjection. For production of transgenic animals, transgenes can be microinjected into fertilized oocytes, or can be incorporated into the genome of embryonic stem cells, and the nuclei of such cells transferred into enucleated oocytes.

When the nucleic acid molecules encoding the humanized heavy and light chains are cloned into separate expression vectors, the vectors can be co-transfected to obtain expression and assembly of intact immunoglobulins. Once expressed, the whole antibodies, their dimers, individual light and heavy chains, or other immunoglobulin forms of the antibodies may be purified according to standard procedures of the art, including ammonium sulfate precipitation, affinity columns, column chromatography, HPLC purification, gel electrophoresis and the like. Substantially pure immunoglobulins of at least about 90 to 95% homogeneity can be prepared for pharmaceutical uses. In another embodiment, substantially pure humanized antibodies of at least about 98 to 99% or more homogeneity can be produced for use in the pharmaceutical formulations and methods.

Thus, also provided is a method of expressing the humanized anti-CD20 antibodies comprising: (a) transforming a host cell with a nucleic acid molecule encoding a humanized anti-CD20 antibody described herein, and (b) culturing the transformed host cells under conditions that allow for the expression of the humanized anti-CD20 antibodies. Known techniques may be used that include a selection marker on the vector so that host cells that express the humanized and chimeric antibodies and the marker can be easily selected.

In one preferred embodiment for producing the humanized anti-CD20 antibodies described herein, transformed duckweed plant or duckweed nodule cultures are used for the expression and the secretion of the antibodies. Genetic techniques for transforming duckweed and optimizing the nucleotide sequence of the expression cassette(s) encoding the antibody are described in U.S. Pat. No. 6,815,184, the entire content of which is hereby incorporated herein by reference.

### Glycosylation

The anti-CD20 antibodies described herein may be glycosylated or unglycosylated. When the anti-CD20 antibodies are glycosylated, the glycan structures that are present may vary as desired. For example, using different host cells for the recombinant production of the humanized anti-CD20 antibodies will vary the glycan structure(s) of the antibodies.

The glycan structures of the antibodies may be also be optimized using known techniques such as RNA interference, antisense, and knockout technologies, etc. in the host cell line. For example, when plant cells and/or plants are used to produce the anti-CD20 antibodies, the native N-glycosylation pattern of the antibodies may be altered by using methods to inhibit the expression of α1,3-fucosyltransferase and β1,2-xylosyltransferase (e.g., using RNA interference constructs). Examples of such technology and plants are described in International Publication No. WO 2007/084926, the entirety of which is incorporated herein.

Thus, in one embodiment, the anti-CD20 antibodies may be prepared in plants such that the N-glycosylation pattern has reduced α1,3-fucose and β1,2-xylose as compared to such antibodies produced in plants without using methods to inhibit the expression of α1,3-fucosyltransferase and β1,2-xylosyltransferase.

In another embodiment, the anti-CD20 antibodies may have a glycosylation pattern that is devoid of fucose and xylose.

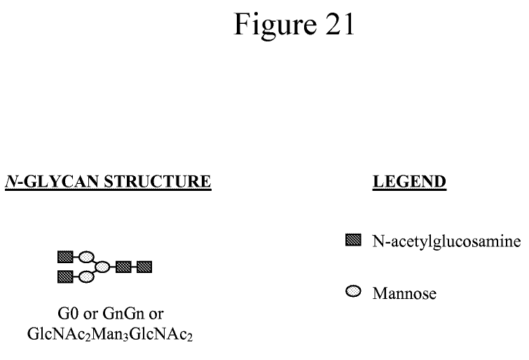

In yet another embodiment, the anti-CD20 antibodies may have an N-glycosylation pattern that is predominantly G0 glycan. “G0 glycan” means the complex N-linked glycan having the structure GlcNAc₂Man₃GlcNAc₂ as shown in FIG. 21, wherein GlcNAc is N-acetylglucosamine and Man is mannose, and wherein a GlcNAc is attached to both the 1,3 mannose arm and the 1,6 mannose arm of the core, which is attached to an amino acid residue of the antibody. Thus, for example, the anti-CD20 antibody composition may have a substantially homogeneous N-glycosylation profile wherein at least 80%, at least 85%, at least 90%, 91%, 92%, 93%, 94%, 95%, 96%, 97%, 98%, or at least 99% of the total amount of the N-glycans in the anti-CD20 antibody composition is represented by the G0 glycan species.

Some immunoglobulins have conserved N-linked glycosylation of the Fc regions of the heavy chains. For example, IgG type immunoglobulins have glycosylated C_{H}2 domains having N-linked oligosaccharides at asparagines 297. Antibodies with reduced fucose content in the oligosaccharides attached to the asparagines at position 297 of the Fc region may enhance the affinity of Fc for FcγRIII, which in turn may increase ADCC of the antibodies.

Thus, the antibodies having an N-glycosylation profile that is predominantly G0 may have increased ADCC activity relative to antibodies produced in plants that do not have inhibited expression or function of α1,3-fucosyltransferase (and thus produce antibodies with more α1,3-fucose).

### Methods of Treatment

The humanized anti-CD20 antibodies of, including antigen-binding fragments thereof, may be used in the treatment of subjects having any disease or disorder associated with CD20-expressing cells, such as normal and malignant B cells expressing CD20 antigen. Thus, the humanized anti-CD20 antibodies are useful for the treatment of B-cell disorders.

By “malignant” B cell is intended any neoplastic B cell, including but not limited to B cells derived from lymphomas including low, intermediate-, and high-grade B cell lymphomas, immunoblastic lymphomas, non-Hodgkin's lymphomas, Hodgkin's disease, Epstein-Barr Virus (EBV) induced lymphomas, and AIDS-related lymphomas, as well as B cell acute lymphoblastic leukemias, myelomas, chronic lymphocytic leukemias, acute myeloblastic leukemias, and the like.

Thus, the humanized anti-CD20 antibodies are useful to treat a number of malignant and non-malignant diseases including autoimmune diseases and related conditions, and CD20 positive cancers including B cell lymphomas and leukemias. Stem cells (B-cell progenitors) in bone marrow lack the CD20 antigen, allowing healthy B-cells to regenerate after treatment and return to normal levels within several months.

Autoimmune diseases or autoimmune related conditions that can be treated using the anti-CD20 antibodies, include arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, responses associated with inflammatory bowel disease (IBD) (Crohn's disease, ulcerative colitis), respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, systemic lupus erythematosus (SLE), lupus (including nephritis, non-renal, discoid, alopecia), juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet's disease, Castleman's syndrome, Goodpasture's Syndrome, Lambert-Eaton Myasthenic Syndrome, Raynaud's syndrome, Sjögren's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliaceus), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimmune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes (also referred to as insulin-dependent diabetes mellitus (IDDM)), Sheehan's syndrome, autoimmune hepatitis, Lymphoid interstitial pneumonitis (HIV), bronchiolitis obliterans (non-transplant) vs NSIP, Guillain-Barré Syndrome, Large Vessel Vasculitis (including Polymyalgia Rheumatica and Giant Cell (Takayasu's) Arteritis), Medium Vessel Vasculitis (including Kawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, coronary artery disease.

CD20 positive cancers that may be treated using the humanized anti-CD20 antibodies described herein include those comprising abnormal proliferation of cells that express CD20 on the cell surface. The CD20 positive B cell neoplasms include CD20-positive Hodgkin's disease, including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); and Hairy cell leukemia. The non-Hodgkins lymphomas include low grade/follicular non-Hodgkin's lymphoma (NHL), small lymphocytic lymphoma (SLL), intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma, and Waldenstrom's macroglobulinemia. Treatments of relapses of these cancers are also contemplated.

In some embodiments, the humanized anti-CD20 antibodies may be used to treat non-Hodgkin's lymphoma (NHL), lymphocyte predominant Hodgkin's disease (LPHD), small lymphocytic lymphoma (SLL), chronic lymphocytic leukemia, rheumatoid arthritis and juvenile rheumatoid arthritis, systemic lupus erythematosus (SLE), Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Raynaud's syndrome, Sjögren's syndrome, and glomerulonephritis.

The humanized anti-CD20 antibodies described herein are useful as a single-agent treatment in, e.g., for relapsed or refractory low-grade or follicular, CD20-positive, B-cell NHL, or can be administered to patients in conjunction with other drugs in a multi drug regimen.

Depending on the indication to be treated and factors relevant to the dosing that a physician of skill in the field would be familiar with, the humanized anti-CD20 antibodies may be administered at a dosage that is efficacious for the treatment of that indication while minimizing toxicity and side effects. For the treatment of a CD20 positive cancer or an autoimmune disease, the therapeutically effective dosage may be in the range of about 100 mg/m² to about 600 mg/m², although higher and lower doses may also be used. In different embodiments, the dosage may be 100 mg/dose, 125 mg/dose, 150 mg/dose, 175 mg/dose, 200 mg/dose, 225 mg/dose, 250 mg/dose, 275 mg/dose, 300 mg, 325 mg/dose, 350 mg/dose, 375 mg/dose, 400 mg/dose, 425 mg/dose, 450 mg/dose, 475 mg/dose, 500 mg/dose, 525 mg/dose, 550 mg/dose, 575 mg/dose, 600 mg/dose. Other dosing amounts, regimens, and intervals may also be used.

In treating disease, the humanized anti-CD20 antibodies may be administered to the patient chronically or intermittently, as determined by the physician of skill in the disease.

In some embodiments, a patient may receive an initial conditioning dose(s) of the antibody followed by a therapeutic dose to alleviate or minimize any adverse events. The conditioning dose(s) will be lower than the therapeutic dose to condition the patient to tolerate higher dosages.

The humanized anti-CD20 antibodies may be administered using any available route of administration, including by intravenous administration (e.g., as a bolus or by continuous infusion over a period of time), or by subcutaneous, intramuscular, intraperitoneal, intracerobrospinal, intra-articular, intrasynovial, intrathecal, or inhalation routes.

In one embodiment, the humanized anti-CD20 antibodies are administered by intravenous infusion with 0.9% sodium chloride solution as an infusion vehicle.

In treating the B cell neoplasms described above, the patient may also be treated with the humanized anti-CD20 antibodies described herein in conjunction with one or more therapeutic agents such as a chemotherapeutic agent in a multidrug regimen. The humanized anti-CD20 antibodies may be administered concurrently, sequentially, or alternating with the chemotherapeutic agent, or after non-responsiveness with other therapy. Standard chemotherapy for lymphoma treatment may include cyclophosphamide, cytarabine, melphalan and mitoxantrone plus melphalan. In another embodiment, the humanized CD20 antibody may be used in conjunction with CHOP, which is one of the most common chemotherapy regimens for treating Non-Hodgkin's lymphoma. The following are the drugs used in the CHOP regimen: cyclophosphamide (brand names cytoxan, neosar); adriamycin (doxorubicin/hydroxydoxorubicin); vincristine (Oncovin); and prednisolone (sometimes called Deltasone or Orasone). In particular embodiments, the humanized anti-CD20 antibodies are administered to a patient in need thereof in combination with one or more of the following chemotherapeutic agents: doxorubicin, cyclophosphamide, vincristine, and prednisolone. In a specific embodiment, a patient suffering from a lymphoma (such as a non-Hodgkin's lymphoma) may be treated with the anti-CD20 antibodies described herein in conjunction with CHOP (cyclophosphamide, doxorubicin, vincristine and prednisone) therapy. In another embodiment, the cancer patient may be treated with the humanized anti-CD20 antibody in combination with CVP (cyclophosphamide, vincristine, and prednisone) chemotherapy.

In treating the autoimmune diseases or autoimmune related conditions described above, the patient may be treated with the humanized anti-CD20 antibodies in conjunction with a second therapeutic agent, such as an immunosuppressive agent (such as in a multi drug regimen). The humanized anti-CD20 antibody may be administered concurrently, sequentially, or alternating with the immunosuppressive agent, or upon non-responsiveness with other therapy.

Immunosuppressive agents for use in adjunct therapy include any substances that act to suppress or mask the immune system of a patient. Such agents include, but are not limited to, substances that suppress cytokine production, down regulate or suppress self-antigen expression, or mask the MHC antigens. Examples of such agents include steroids such as glucocorticosteroids (e.g., prednisone, methylprednisolone, and dexamethasone); 2-amino-6-aryl-5-substituted pyrimidines, azathioprine; bromocryptine; glutaraldehyde; anti-idiotypic antibodies for MHC antigens and MHC fragments; cyclosporin A; cytokine or cytokine receptor antagonists including anti-interferon-γ, -β, or -α antibodies; anti-tumor necrosis factor-α antibodies; anti-tumor necrosis factor-β antibodies; anti-interleukin-2 antibodies and anti-IL-2 receptor antibodies; anti-L3T4 antibodies; heterologous anti-lymphocyte globulin; pan-T antibodies; soluble peptide containing a LFA-3 binding domain; streptokinase; TGF-β; streptodornase; deoxyspergualin; rapamycin; T-cell receptor; T-cell receptor fragments; and T cell receptor antibodies.

For the treatment of rheumatoid arthritis, the patient may be treated with a humanized anti-CD20 antibody in conjunction with any one or more of the following drugs: DMARDS (disease-modifying anti-rheumatic drugs (e.g., methotrexate)), NSAI or NSAID (non-steroidal anti-inflammatory drugs), HUMIRA™ (adalimumab; Abbott Laboratories), ARAVA® (leflunomide), REMICADE® (infliximab; Centocor Inc., of Malvern, Pa.), ENBREL (etanercept; Immunex, Wash.), and COX-2 inhibitors.

### Formulations

Therapeutic formulations of the humanized anti-CD20 antibodies may be prepared for storage or use by mixing an antibody having the desired degree of purity with optional pharmaceutically acceptable carriers, excipients, or stabilizers in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations herein may also contain more than one active compound as necessary for the particular indication being treated. For example, it may be desirable to further provide a cytotoxic agent, chemotherapeutic agent, cytokine, or immunosuppressive agent. The effective amount of such other agents depends on the amount of antibody present in the formulation, the type of disease or disorder or treatment, and other factors discussed above.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

Sustained-release preparations may also be prepared comprising the humanized anti-CD20 antibodies. Suitable examples of sustained-release preparations include semi-permeable matrices of solid hydrophobic polymers containing the antagonist, which matrices are in the form of shaped articles, e.g. films, or microcapsules. Examples of sustained-release matrices include, but are not limited to, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers, and poly-D-(−)-3-hydroxybutyric acid.

### Conjugated Antibodies and Other Methods

The humanized anti-CD20 antibodies may be conjugated with one or more therapeutic or diagnostic agents.

The humanized antibodies described herein may be used in therapeutic methods and diagnostic methods. Accordingly, the humanized antibodies may be administered alone as a naked antibody or administered as a multimodal therapy, temporally according to a dosing regimen, but not necessarily conjugated to a therapeutic agent. In one embodiment, the efficacy of the naked humanized and chimeric antibodies can be enhanced by supplementing naked antibodies with one or more other naked antibodies, i.e., monoclonal antibodies to specific antigens, such as CD4, CD5, CD8, CD14, CD15, CD19, CD21, CD22, CD23, CD25, CD33, CD37, CD38, CD40, CD40L, CD46, CD52, CD54, CD74, CD80, CD126, B7, MUC1, Ia, HM1.24, or HLA-DR, tenascin, VEGF, PlGF, an oncogene, an oncogene product, or a combination thereof with one or more immunoconjugates of anti-CD20, or antibodies to these recited antigens, conjugated with therapeutic agents, including drugs, toxins, immunomodulators, hormones, therapeutic radionuclides, etc., with one or more therapeutic agents, including drugs, oligonucleotides, toxins, immunomodulators, hormones, therapeutic radionuclides, etc., administered concurrently or sequentially or according to a prescribed dosing regimen, with the humanized antibodies.

Immunoconjugates may be administered for diagnostic and therapeutic uses in B cell lymphomas, autoimmune diseases, transplant rejections, and other disease or disorders. Thus, the humanized anti-CD20 antibodies may be conjugated to a cytotoxic agent such as a toxin or a radioactive isotope. In some embodiments, the toxin is calicheamicin, a maytansinoid, a dolastatin, auristatin E and analogs or derivatives thereof.

A wide variety of diagnostic and therapeutic agents can be conjugated to the humanized anti-CD20 antibodies. Such therapeutic agents can also be used for administration separately with the naked antibody as described above. Therapeutic agents include, for example, chemotherapeutic drugs such as vinca alkaloids, anthracyclines, epidophyllotoxin, taxanes, antimetabolites, alkylating agents, antikinase agents, antibiotics, Cox-2 inhibitors, antimitotic, antiangiogenic and apoptotoic agents, particularly doxorubicin, methotrexate, taxol, CPT-11, camptothecans, and others from these and other classes of anticancer agents, and the like. Other useful cancer chemotherapeutic drugs for the preparation of immunoconjugates and antibody fusion proteins include nitrogen mustards, alkyl sulfonates, nitrosoureas, triazenes, folic acid analogs, COX-2 inhibitors, pyrimidine analogs, purine analogs, platinum coordination complexes, hormones, and the like.

Additional toxins for use in conjugation with the humanized antibodies include any pharmaceutically acceptable toxins, and include, but are not limited to DNA damaging agents, inhibitors of microtubule polymerization or depolymerization and antimetabolites. Classes of cytotoxic agents include, for example, the enzyme inhibitors such as dihydrofolate reductase inhibitors, and thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins and differentiation inducers.

The humanized anti-CD20 antibodies may also be conjugated with a radioactive isotope. For example, for selective destruction of a tumor, the antibody may comprise a highly radioactive atom. A variety of radioactive isotopes are available for the production of radioconjugated anti-CD20 antibodies. When the conjugate is to be used for diagnosis, it may comprise a radioactive atom for scintigraphic studies, or a spin label for nuclear magnetic resonance (NMR) imaging.

The labels may be incorporated into the conjugate using any method. For example, the peptide may be biosynthesized or may be synthesized by chemical amino acid synthesis using suitable amino acid precursors involving, for example, fluorine-19 in place of hydrogen.

An oligonucleotide, such as an antisense molecule, may also be conjugated to or be administered with the humanized anti-CD20 antibodies.

The following examples are offered by way of illustration and not by way of limitation.

### EXAMPLES

### Example 1

### Preparation of Humanized 1F5 Antibodies

Chimeric expression vectors were prepared using the heavy and light chain sequences of the mouse antibody 1F5 (Press et al. Blood, 69(2):584-591 (1987)). Genbank accession number AY058906 provides the sequence of 1F5 VK, the light chain of mouse antibody 1F5. This VK uses the AJ231219 germline V gene to whose DNA sequence it is 98% identical. GenBank AY058907 accession number is the sequence of 1F5 VH, the heavy chain of mouse antibody 1F5. This VH uses the AC090843 germline V gene to whose DNA sequence it is 95% identical.

The construction of the chimeric expression vectors included adding a suitable leader sequence to the mouse VH and VK, preceded by a HindIII restriction site and a Kozak sequence. The Kozak sequence ensures efficient translation of the variable region sequence. It defines the correct AUG codon from which a ribosome can commence translation. An important base in the Kozak sequence is the adenine at position −3, upstream of the AUG start. The human 1210 VK leader sequence was predicted to cut correctly by signal protease when contiguous with 1F5 VK. The human 1210 VH leader sequence was predicted to cut correctly by signal protease when contiguous with 1F5 VH. The appropriate 1210 leader sequences were therefore cloned upstream (5′) of the 1F5 VH and VK coding regions.

Construction of the chimeric expression vectors also included introducing a 5′ fragment of the human γ1 constant region, up to a natural ApaI restriction site, contiguous with the 3′ end of the J region of 1F5. The human γ1 constant region is encoded in the expression vector, downstream of the inserted VH sequence but, unlike the kappa construct, it lacks a V-C intron. For the kappa chain, the natural splice donor site and a BamHI site is added downstream of the V region. The splice donor sequence facilitates the splicing out of the kappa V-C intron, which is useful for in-frame attachment of VK to the kappa constant region.

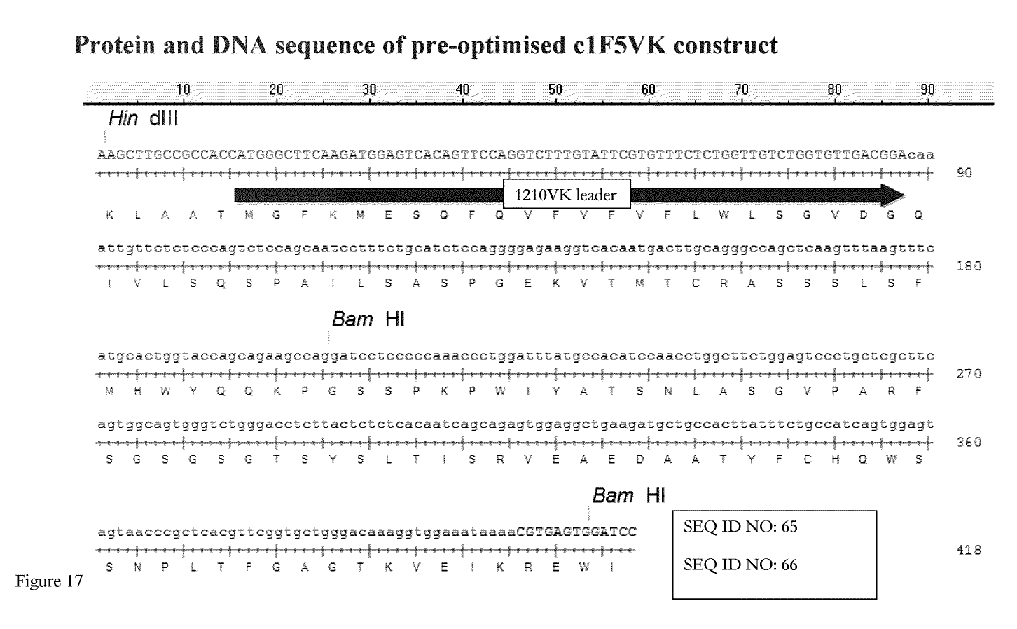

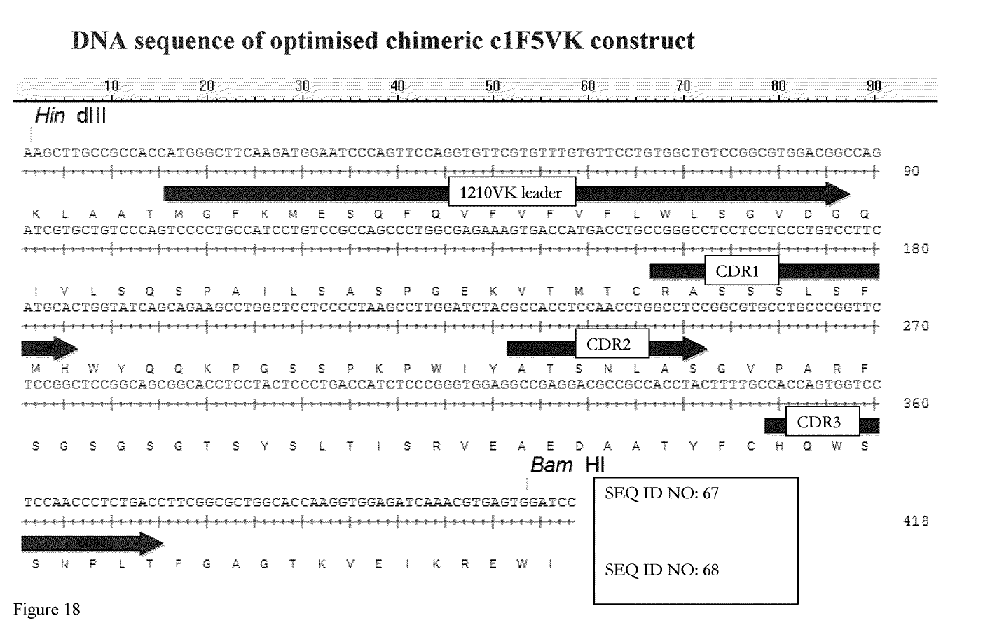

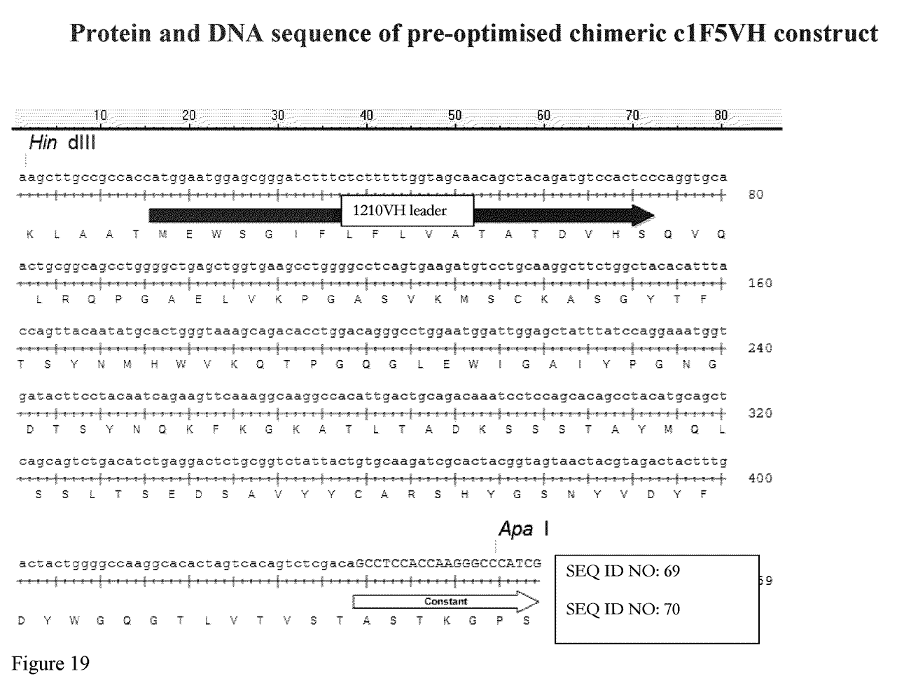

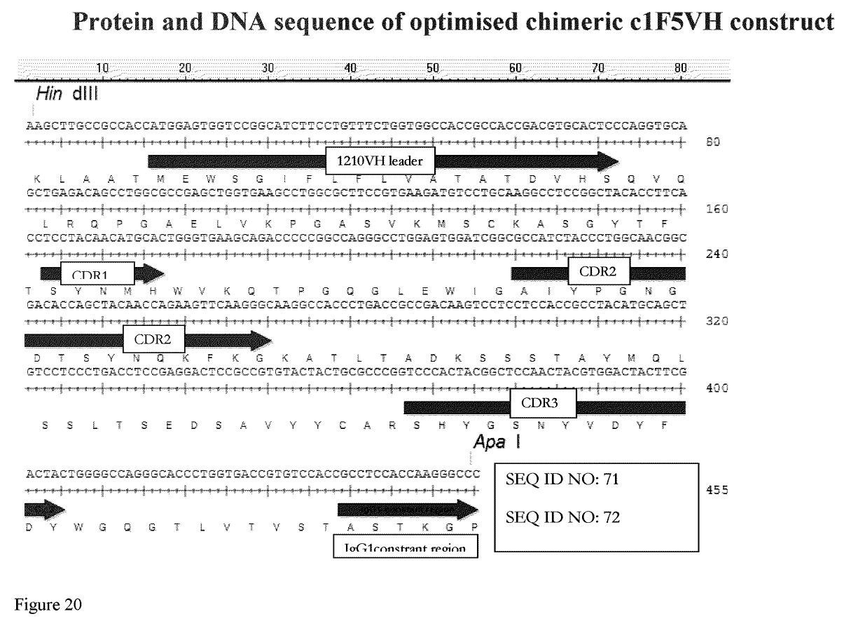

The mouse 1F5 VH and VK genes were analyzed to identify any extra restriction sites which may interfere with the subcloning. One restriction site was found in the pre-optimized 1F5VK sequence (FIG. 17) but none were identified in 1F5VH sequence (FIG. 19). These DNA sequences were optimized for expression, by removing internal TATA-boxes, chi-sites and ribosomal entry sites, AT-rich or GC-rich sequence stretches; ARE, INS, CRS sequence elements; repeat sequences and RNA secondary structures; (cryptic) splice donor and acceptor sites, branch points; by adapting codon usage to the codon bias of Cricetulus griseus/homo sapiens; and by increasing the GC content to improve mRNA stability. The optimized c1F5VK (FIG. 18) and c1F5VH (FIG. 20) DNA construct sequences were synthesized and cloned into cloning vector pGA4 (Geneart AG, Regensburg, Germany). The VK and VH inserts were excised with (BamHI+HindIII) or (HindIII+ApaI) respectively. The excised fragments were then gel purified, ligated into pKN100 or pG1D200, respectively, which had been cut similarly, and phosphatase digested. The ligated constructs were transformed into competent TOP10 bacteria. Expression clone colonies were screened by PCR for the presence of an insert of the correct size using primers g10545 (5′-TGTTCCTTTCCATGGGTCTT) (SEQ ID NO:75) and g23070 (5′-GTGTGCACGCCGCTGGTC) (SEQ ID NO:76). Minipreps were generated of the correct expression clones and used to transform HEK293T cells.

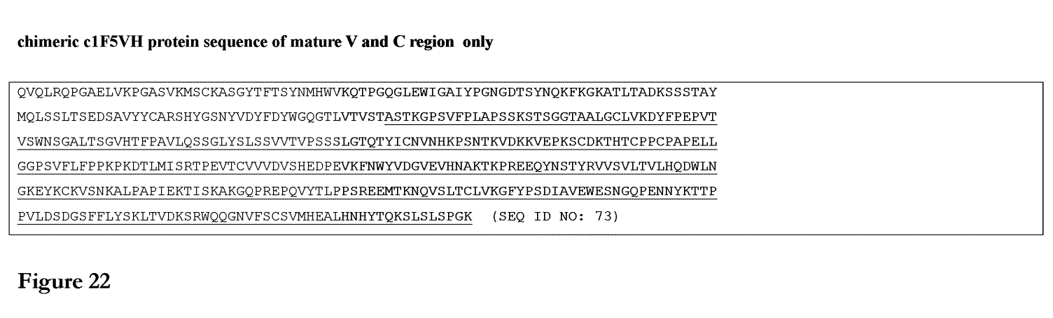

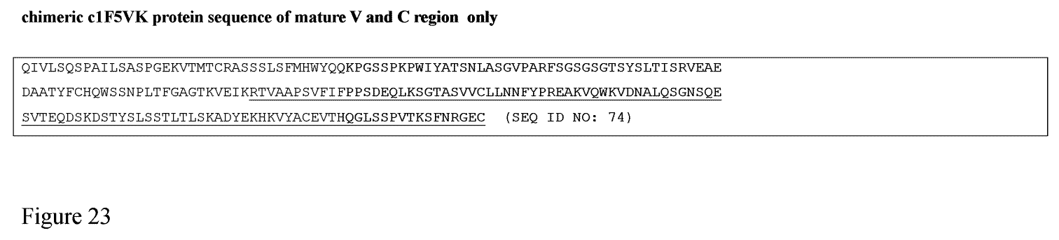

FIG. 22 shows the amino acid sequence of the heavy chain of the mature chimeric 1F5 antibody with constant region underlined. FIG. 23 shows the amino acid sequence of the light chain of the mature chimeric 1F5 antibody with constant region underlined.

The chimeric heavy and light chain sequences of the murine monoclonal antibody 1F5 were then humanized by identifying appropriate human FR sequences from sequence comparisons to human and mouse immunoglobulins. The protein sequences of human and mouse immunoglobulins from the International Immunogenetics Database 2006 (Lefranc, M. P., Nucl. Acids Res., 31:307-310 (2003)) and the Kabat Database (Kabat, E. A., et al., NIH National Technical Information Service, 1-3242 (1991)) Release 5 of Sequences of Proteins of Immunological Interest (last update 17 Nov. 1999) were used to compile a database of immunoglobulin protein sequences in Kabat alignment. The database contained 9322 human VH and 2689 human VK sequences. A sequence analysis program, Gibbs, was used to query the human VH and VK databases with 1F5 VH and VK protein sequences.

### Generation of 1F5RHA and 1F5RHB

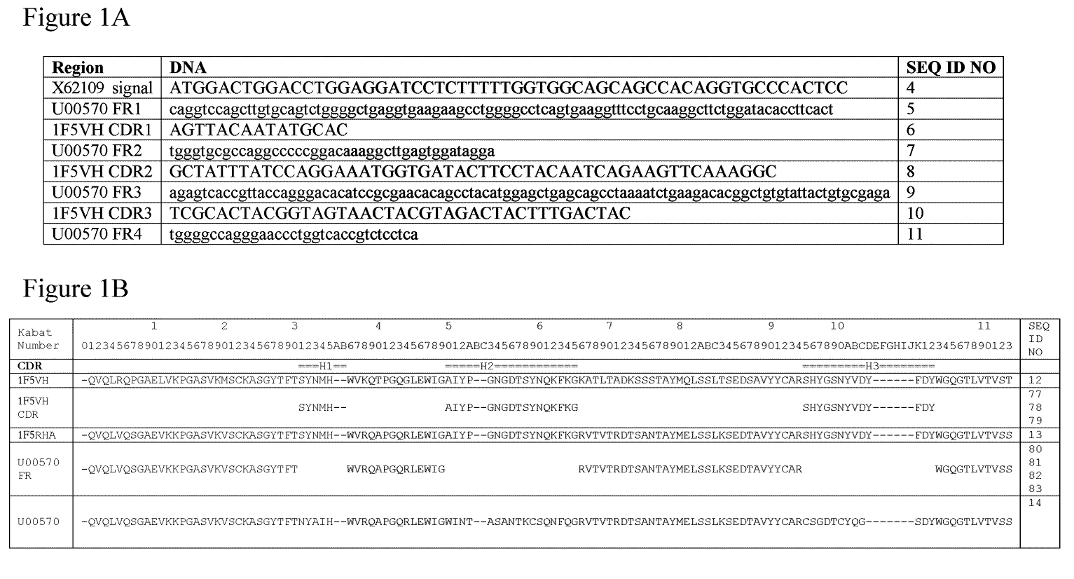

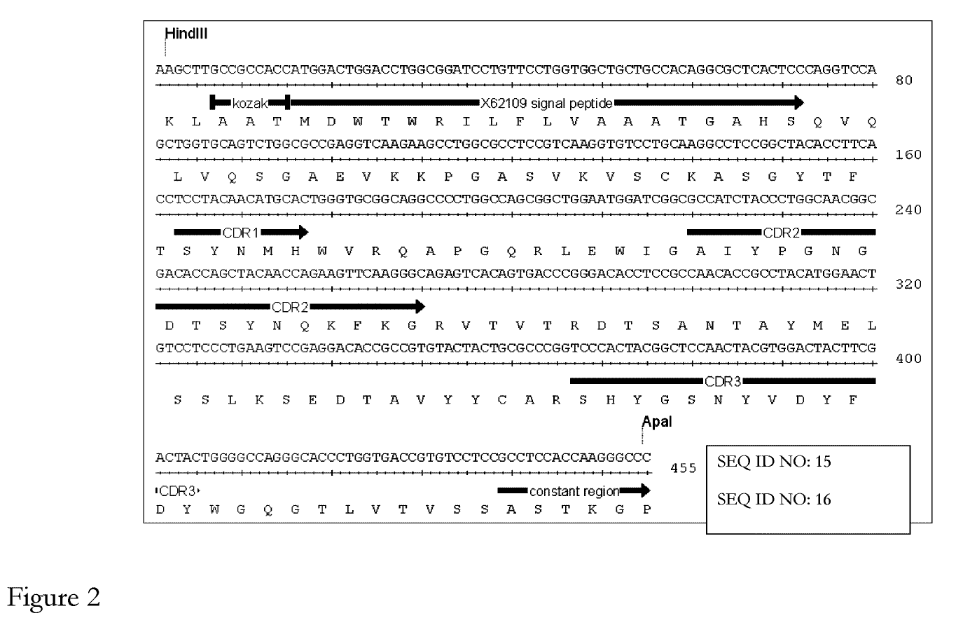

To generate a humanized VH sequence, human VH sequences in the database were identified that had the highest identity to 1F5VH at Vernier, Canonical and VH-VK Interface (VCI) residues, located within the V-region framework. Of the VH sequences identified, accession number U00570 was chosen as the human FR donor for 1F5RHA. The human germline V-gene most identical to U00570 is the VHI family gene X62109 (VI-3B), from which the signal peptide was extracted. The SignalP algorithm predicted that it would cut appropriately with signal peptidase. FIG. 1A shows the sequences used in the generation of the DNA sequence 1F5RHAss from the natural DNA sequences of the X62109 signal, 1F5VH CDRs and U00570 FR. CDRs 1, 2 and 3 from 1F5VH were grafted into the acceptor FR of U00570 to generate 1F5RHA (FIG. 1B). The DNA sequence was optimized by silent mutagenesis (GeneArt) to remove internal TATA-boxes, chi-sites and ribosomal entry sites, AT-rich or GC-rich sequence stretches, ARE, INS, CRS sequence elements, repeat sequences and RNA secondary structures (cryptic) splice donor and acceptor sites and branch points. The optimized 1F5RHA construct DNA and protein sequence is shown in FIG. 2. This construct contains no mutations in the framework region.

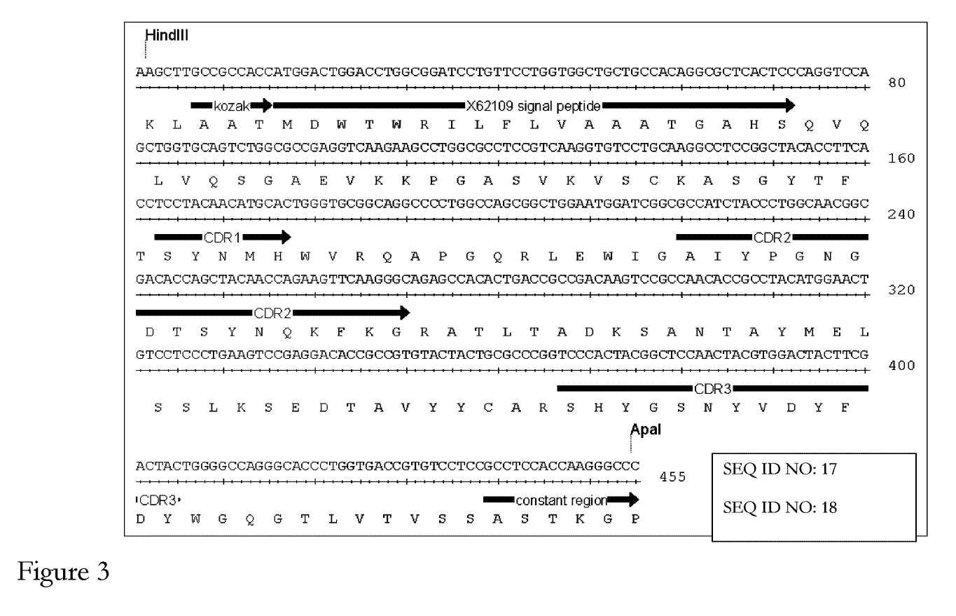

A second construct, 1F5RHB (FIG. 3), was based on the 1F5RHA sequence, to contain 4 back-mutations at the non-conserved vernier residues as follows: V67A; V69L; R71A and T73K (Kabat numbering) (FIG. 10A).

### Generation of 1F5RKA11 and 1F5RKA12

Humanized VK sequences were generated based on sequence comparisons to human VK sequences with the highest identity to 1F5VK at VCI residues, located within the V-region framework. From sequence comparisons, there were no human VK genes identified with the same length of CDR1 as 1F5VK (10 amino acids). Two groups of human FRs with the closest CDR1 lengths (11 and 12 amino acids) were then analyzed that had identical lengths of CDR 2 and 3 as 1F5VK.

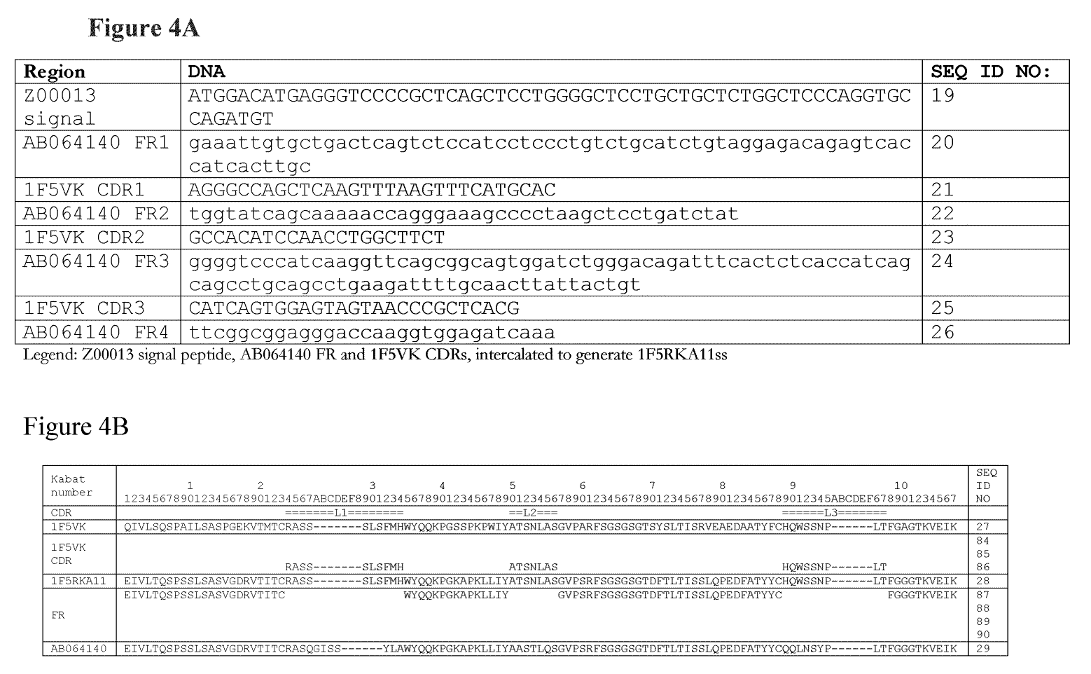

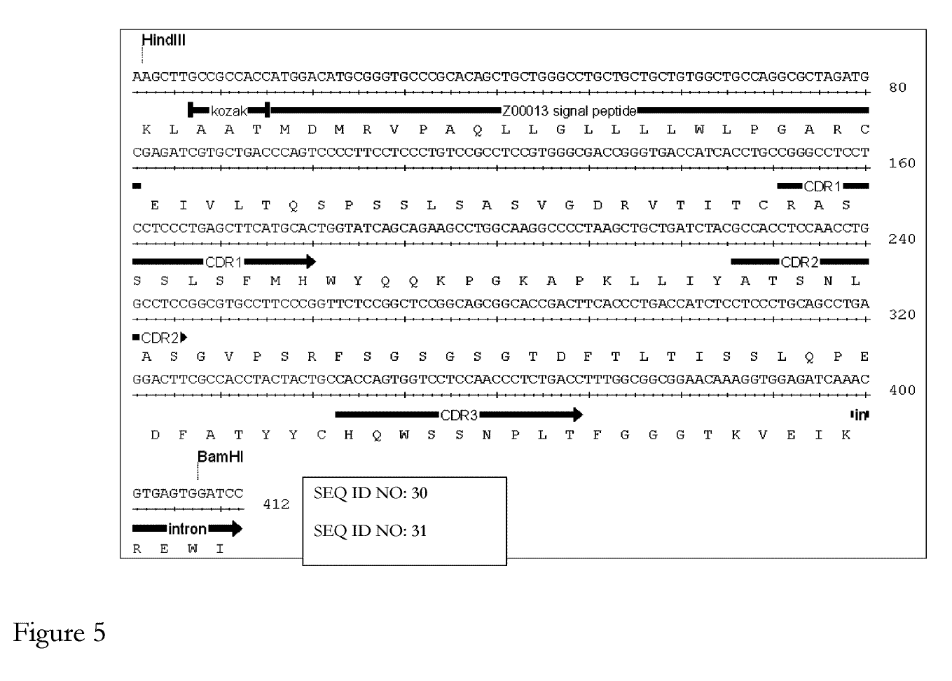

For the 11 amino acid CDR1 sequences, Accession number AB064140 was chosen as the FW donor on which to base 1F5RKA11. FIG. 4A shows the generation of 1F5RKA11ss from the natural DNA sequences of 1F5 VK, the human VK sequence AB064140 and the human Z00013 signal sequence. This sequence also was optimized to generate the 1F5RKA11 construct (FIG. 5), which contains no FR mutations. FIG. 4B provides the amino acid sequences of the FR and CDRs used to generate 1F5RKA11.

CDRs 1, 2 and 3 from 1F5VK were grafted into the acceptor FR of human AB064140 to generate 1F5RKA11. The nearest germline V-gene to AB064140 is Z00013 (Vd/L8), from which the signal peptide was obtained. The SignalP algorithm predicted appropriate cutting of this signal peptide.

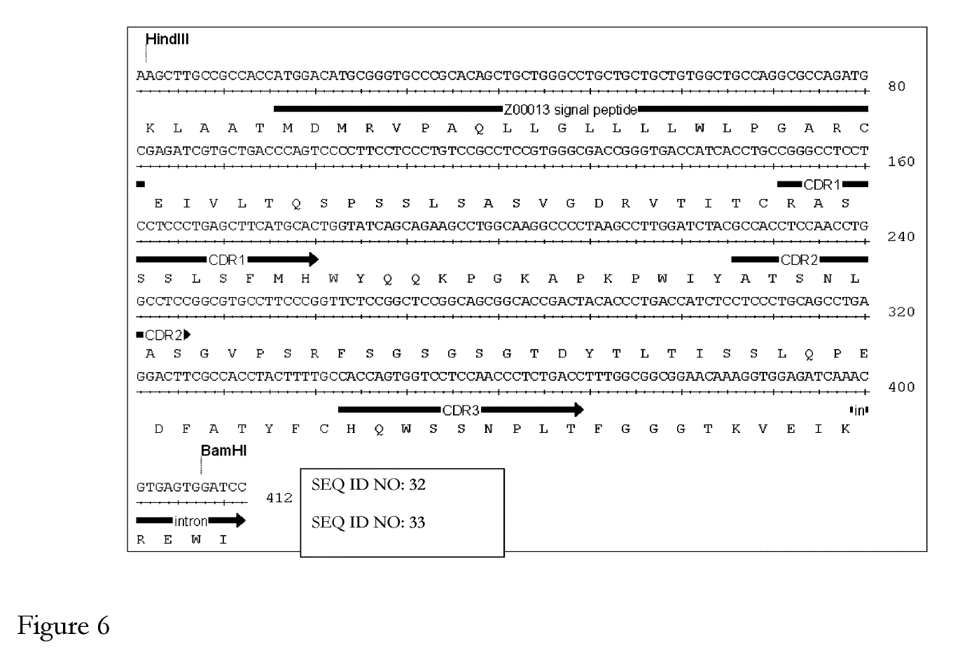

A second construct, 1F5RKB11 (FIG. 6), was generated from 1F5RKA11 by introducing four back mutations at the three non-conserved vernier residues: L46P; L47W and F71Y (Kabat numbering); together with VH/VK interface residue Y87F (Kabat numbering) (FIG. 10B). Further versions: 1F5RKB11; 1F5RKD11; 1F5RKE11 and 1F5RKF11 were based on 1F5RKB11, each containing a different reversal of one of these 4 mutations (FIG. 10B).

A second set of humanized VK sequences were also prepared based on a human kappa sequence with CDR1 sequences containing 12 amino acids. Accession number AY263415 was selected as the FR donor on which to base 1F5RKA12.

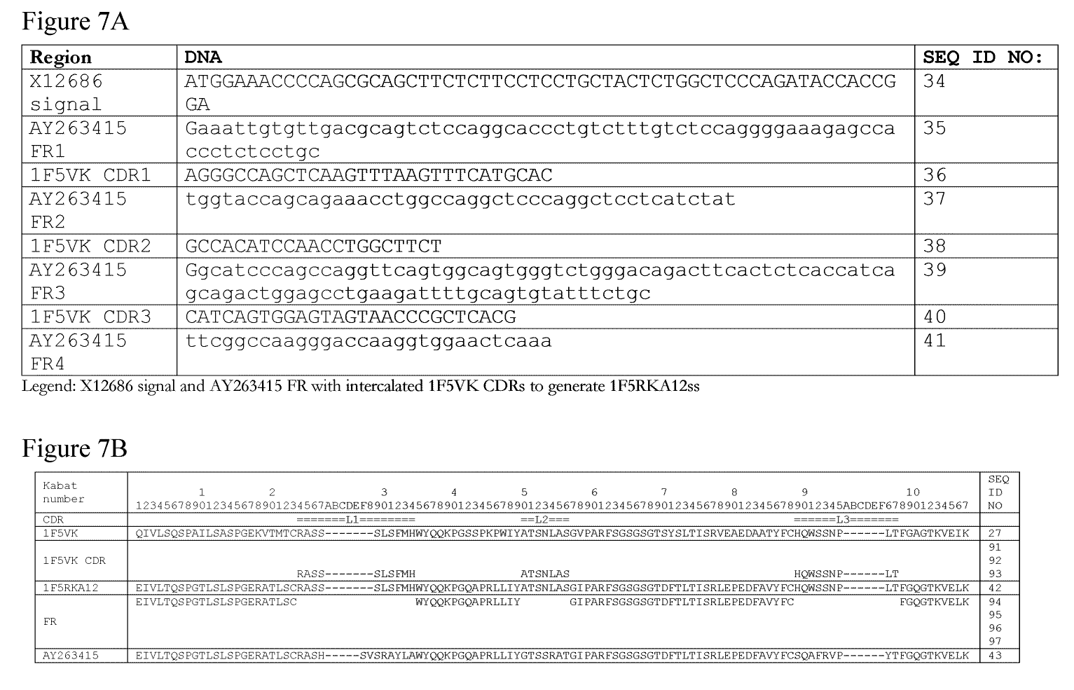

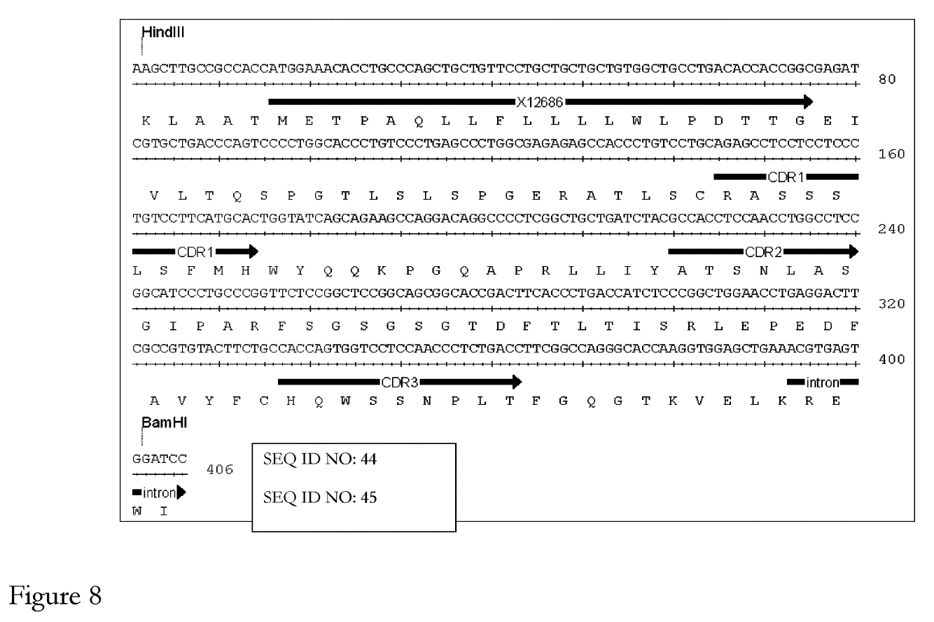

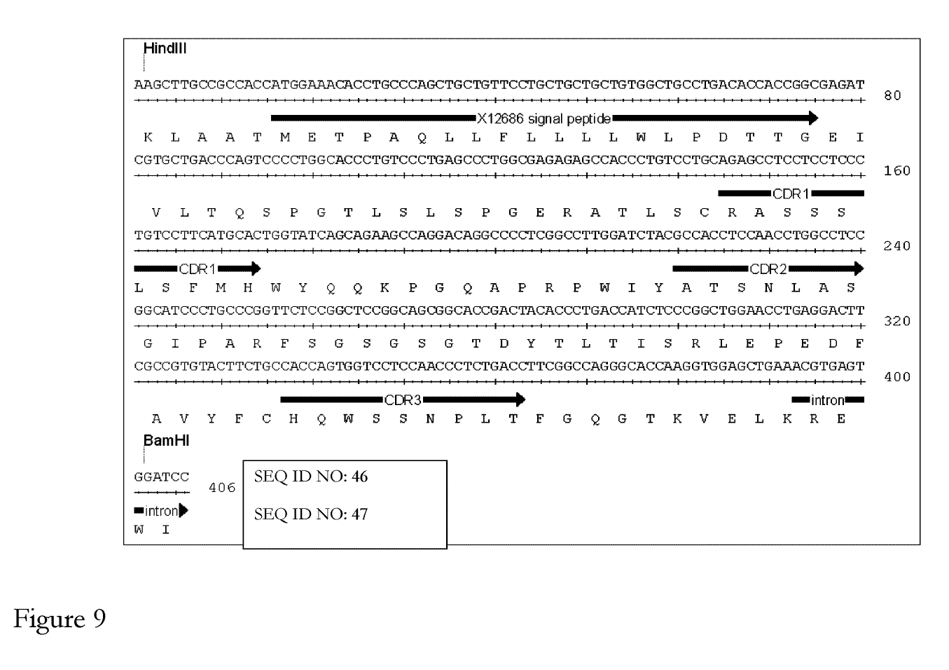

FIG. 7A shows the nucleic acid sequences used to generate 1F5RKA12ss from the natural DNA sequence of 1F5 VK, the human VK sequence AY263415 and the human X12686 signal sequence. CDRs 1, 2 and 3 from 1F5VK were grafted into the acceptor FR of human AY263415 to generate 1F5RKA12 (FIG. 7B). The nearest germline V-gene to accession AY263415 is the VKIII family member, X12686 (A27), from which the signal peptide was obtained. The SignalP algorithm predicted appropriate cutting of this signal peptide. The optimized 1F5RKA12 construct DNA and protein sequence is shown in FIG. 8. This construct contains no FR mutations. A second construct 1F5RKB12 (FIG. 9) was generated from 1F5RKA12 by introducing three back mutations at the three non-conserved vernier residues: L46P; L47W and F71Y (Kabat numbering). Further versions: 1F5RKC12; 1F5RKD12 and 1F5RKE12 were based on 1F5RKB12, each containing a different reversal of one of these 3 mutations.

Expression vectors were prepared that included, for the heavy chains, a human γ1 constant region attached to the variable region, and for the light chains, a kappa constant region attached to the variable region.

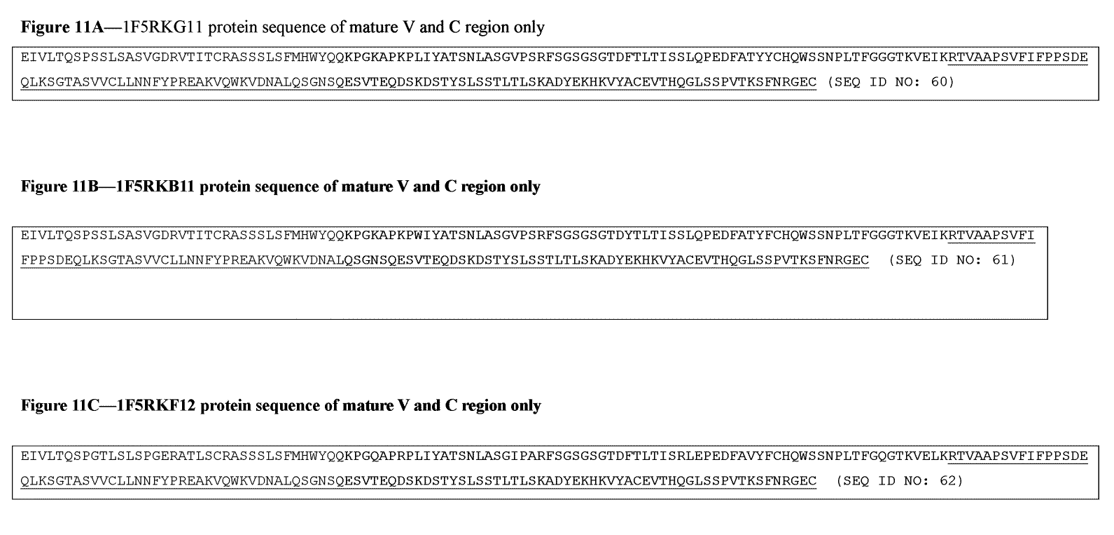

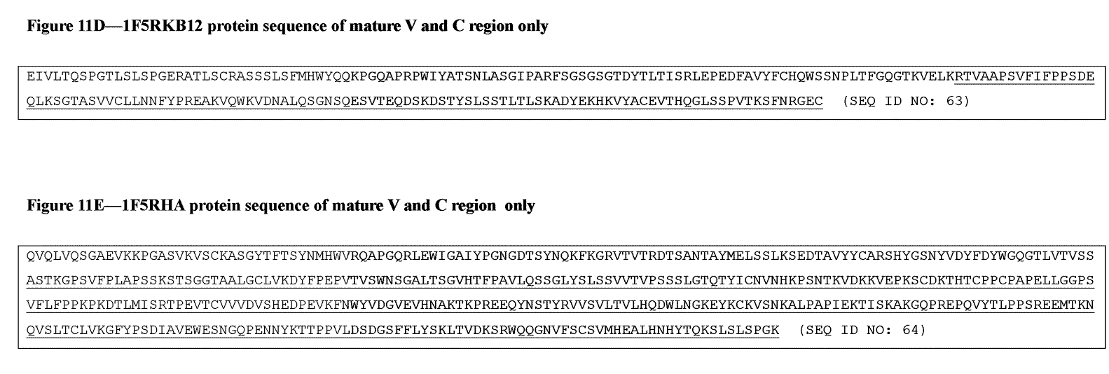

FIGS. 11A-E provide the amino acid sequences of the mature variable and constant regions from 1F5RKG11, 1F5RKB11, 1F5RKF12, 1F5RKB12, and 1F5RHA proteins, respectively. FIGS. 22 and 23 provide the amino acid sequences for a chimeric protein sequence of mature variable and constant regions for c1F5VH and c1F5VK, respectively. The constant regions of the amino acid sequences are underlined.

The expression plasmid preparations encoding (humanized or chimeric) heavy and light chains were used to transfect HEK293t cells. For transfection, cells were initially grown in DMEM plus GlutaMax supplemented with 10% FCS, penicillin, and streptomycin in a flask and incubated in a CO₂-gassed cell culture chamber. The cell cultures were split 1:3 to every two days or 1:4 or 1:5 every 3-4 days. Light trypsinisation was used to detach cells from flasks during passaging. One day before transfection, 6-well plates were prepared by adding 2 ml of culture media to each well, followed by the addition of cells (2×10⁵ cells/well). Cells were checked the following day to ensure at least 80% confluency and the media was replaced with 2 ml/well. For transfection, 96 μl of OPtiPRO SFM was aliquoted into a sterile 1.5 ml tube and 6 μl of Fugene 6 was added directly to the media, and incubated at room temperature for 5 minutes. DNA (1 μg total; 0.5 μg heavy chain and 0.5 μg light chain vector) was added and incubated at room temperature for 15 minutes. A mixture of the Fugene 6, DNA, and OptiPro SFM mixture was added, dropwise, around the well, and the plates were then incubated for 4 days in a cell culture incubator. The conditioned medium was harvested after 4 days, IgG was quantified, and antigen-binding assays performed.

IgG in these conditioned media was measured by ELISA to measure Raji cell binding (Example 2). The concentrations of IgG1κ antibody in all transfected 293 cell-conditioned media used are shown in Table 1. The humanized and chimeric antibodies were expressed at good to excellent levels, and were used for the further Examples 2-4 below.

TABLE 1

| | |
|---|---|
| Constructs used | |
| for 293 transfection | IgG |

| | | |
|---|---|---|
| VH | VK | ng/ml |

| | | | |
|---|---|---|---|
| chimeric | chimeric | 3685 | 5580 |
| chimeric | chimeric | 4335 | 5084 |
| chimeric | chimeric | 8714 | 8479 |
| chimeric | KB11 | 847 | 1576 |
| chimeric | KC11 | 4603 | 6381 |
| chimeric | KD11 | 2352 | 4940 |
| chimeric | KE11 | 1537 | 1543 |
| chimeric | KF11 | 1730 | 1730 |
| chimeric | KB12 | 813 | 1062 |
| chimeric | KC12 | 1861 | 1498 |
| chimeric | KD12 | 2772 | 2684 |
| chimeric | KE12 | 2964 | 2964 |
| RHA | chimeric | 3222 | 4215 |
| RHB | chimeric | 4876 | 3306 |
| RHA | chimeric | 6291 | 6895 |
| RHA | RKB12 | 1960 | 2146 |
| RHA | RKC12 | 447 | 494 |
| RHA | RKD12 | 4789 | 4651 |
| RHA | RKE12 | 2517 | 2375 |
| RHA | RKF12 | 3773 | 3394 |
| RHA | RKB11 | 6258 | 2267 |
| RHA | RKC11 | 6258 | 6609 |
| RHA | RKD11 | 8600 | 8366 |
| RHA | RKE11 | 9182 | 7957 |
| RHA | RKF11 | 8517 | 5529 |
| RHA | RKG11 | 8516 | 8184 |

### Example 2

### Raji Binding by Humanized 1F5 Antibodies

Assays were performed to measure binding of the chimeric and humanized 1F5 antibodies to Raji cells (a human B cell line expressing CD20). Raji cells (80 μl; 1-2×10⁶ cells/ml in 24 ml with fresh growth medium diluted with 0.11 vol of 10×PBS (MPBS) and 8 ml 8% paraformaldehyde (in PBS)) were added to 96-well poly-D-lysine-coated plate and centrifuged at 2500 rpm (Beckman 6L) at 25° C. for 60 minutes. The pellets were washed 4 times with 400 μl PBS, Tween 20 (0.02% v/v). 50 μl of MPBS was added to each well after the final wash, and incubated for 1 hour at room temperature, and the blocking buffer was then discarded. Serial dilutions of the 1F5 antibodies were prepared in MPBS in a separate 96-well plate, transferred to the Raji-cell coated plate and incubated at room temperature for 1 hour. The antibody solution was then discarded and the plates were washed 6 times with 400 μl PBS buffer. Goat anti-human kappa (Sigma A7164) was prepared by diluting the stock antibody solution 1:2000 in MPBS, added to the washed wells, and incubated again at room temperature for 1 hour. The plates were again washed 6 times in 400 μl PBS buffer. 100 μl of K-blue TMB solution was added to each well. The reactions were stopped by added 50 μl red stop after 30 minutes, and the absorbance was read at 650 nm.

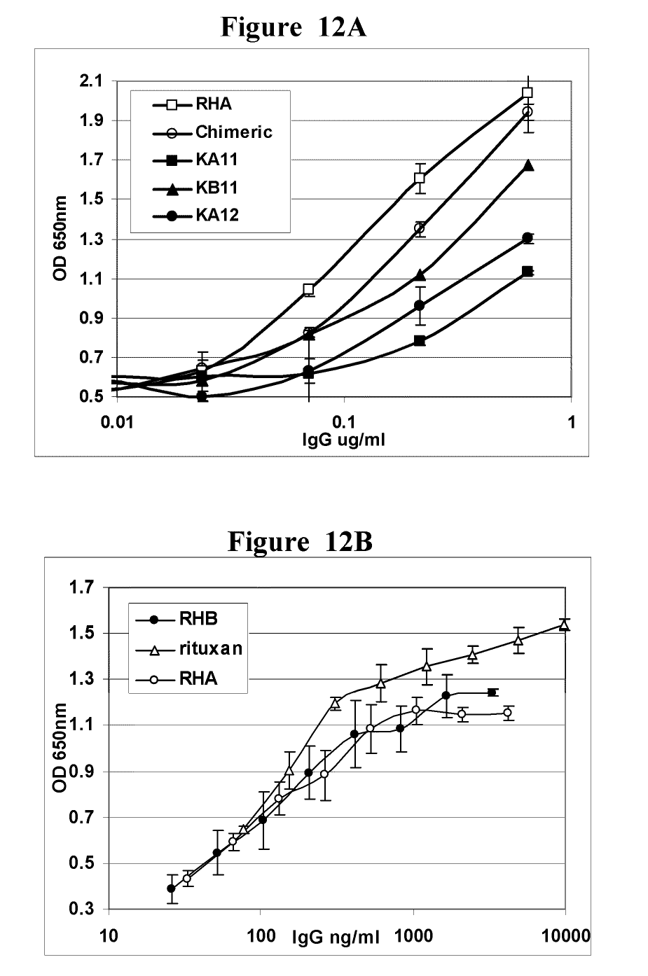

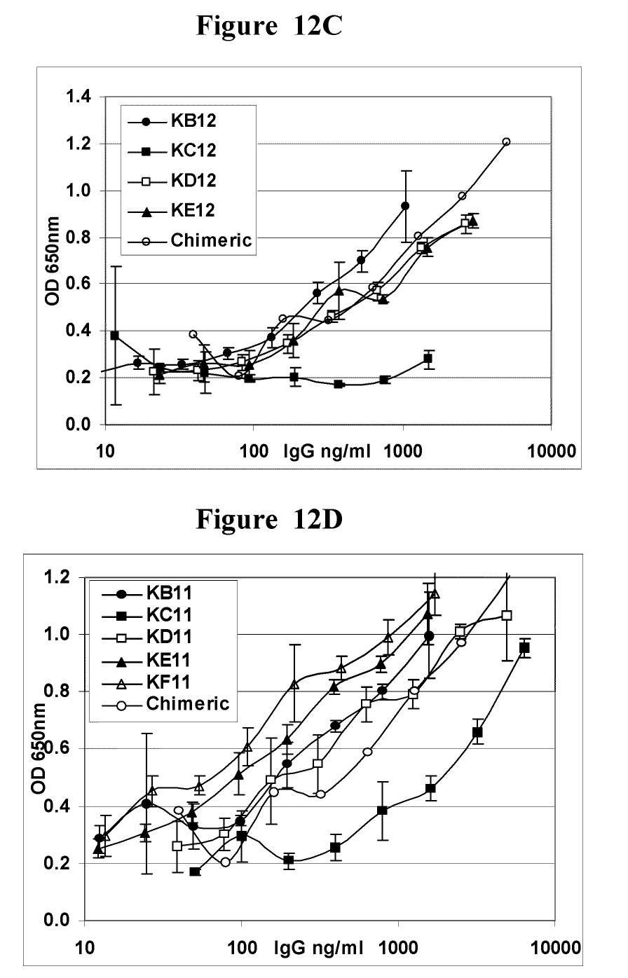

As illustrated in FIG. 12, the binding to Raji cells of antibodies encoded by chimeric c1F5VH, in association with humanized VKs: 1F5RKA11; 1F5RKA12; 1F5RKC11 or 1F5RKC12 was reduced compared with that of the chimeric c1F5 antibody (FIGS. 12A, 12C, and 12D). In contrast, the Raji cell binding by antibodies encoded by chimeric c1F5VK in combination with either 1F5RHA or 1F5RHB was indistinguishable, and similar to that of Rituxan (FIG. 12B). Other VK versions: 1F5RKB11; 1F5RKD11; 1F5RKE11; 1F5RKF11 (FIG. 12D); 1F5RKB12; 1F5RKD12; 1F5RKE12; (FIG. 12C), in combination with chimeric c1F5VH, bound to Raji cells similarly to, or better than, the chimeric antibody.

In light of the low binding potency of the 1F5RKC11 and 1F5RKC12 versions, in which the vernier L46P backmutation (Kabat numbering) was reversed, 1F5RKG11 and 1F5RKF12 were designed (FIGS. 10 B and C, respectively) based on 1F5RKA11 or 1F5RKA12, respectively, with the sole introduction of the L46P back mutation (Kabat numbering).

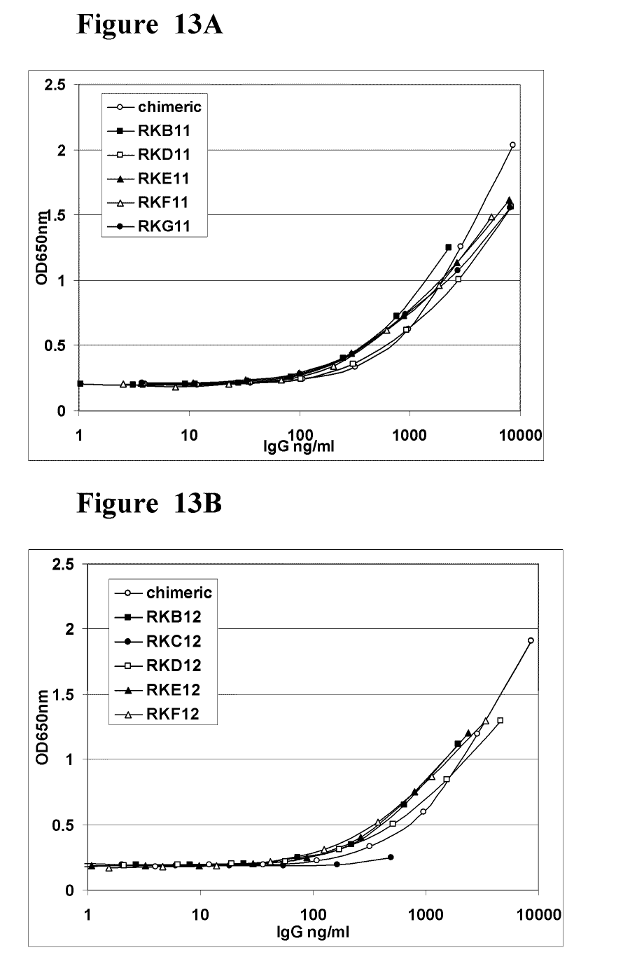

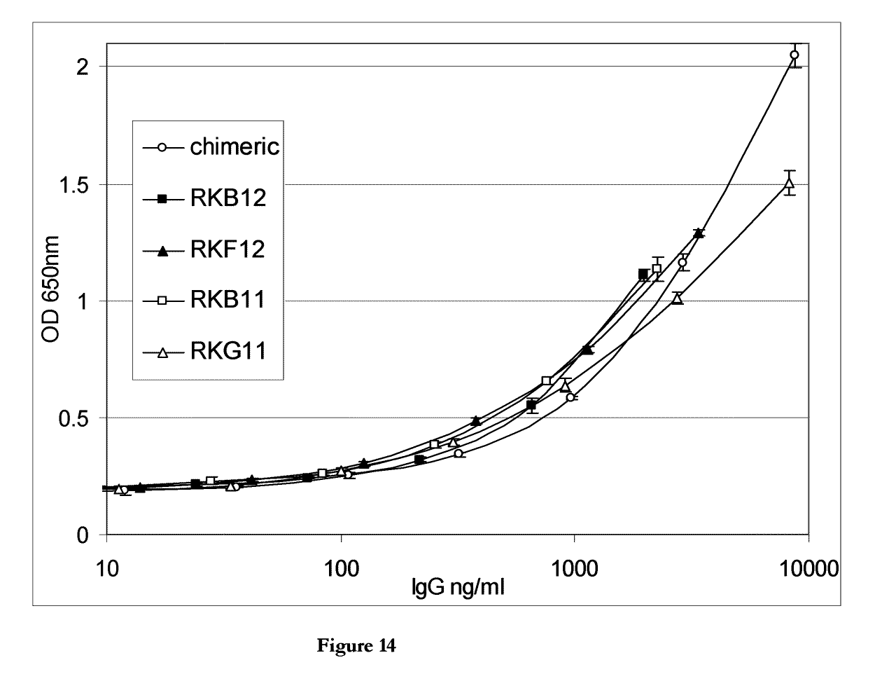

Additional assays (FIGS. 13-15) investigated fully humanized antibodies encoded by 1F5RHA together with various humanized kappa chains. FIGS. 13A-B show that the Raji cell binding by fully humanized antibodies encoded by 1F5RHA, in association with all kappa chain constructs except the 1F5RKC versions, is similar to that of the chimeric antibody. The Raji cell binding by antibodies encoded by the kappa constructs 1F5RKC12 and 1F5RKC11 (not shown) was again poor. FIG. 14 confirms that the Raji cell binding potency of antibody encoded by 1F5RHA in association with 1F5RKF12 appears to be slightly superior to that of the antibody encoded by 1F5RKG11, and that their potencies are similar to that of the chimeric antibody.

### Example 3

### FACS Binding Assay

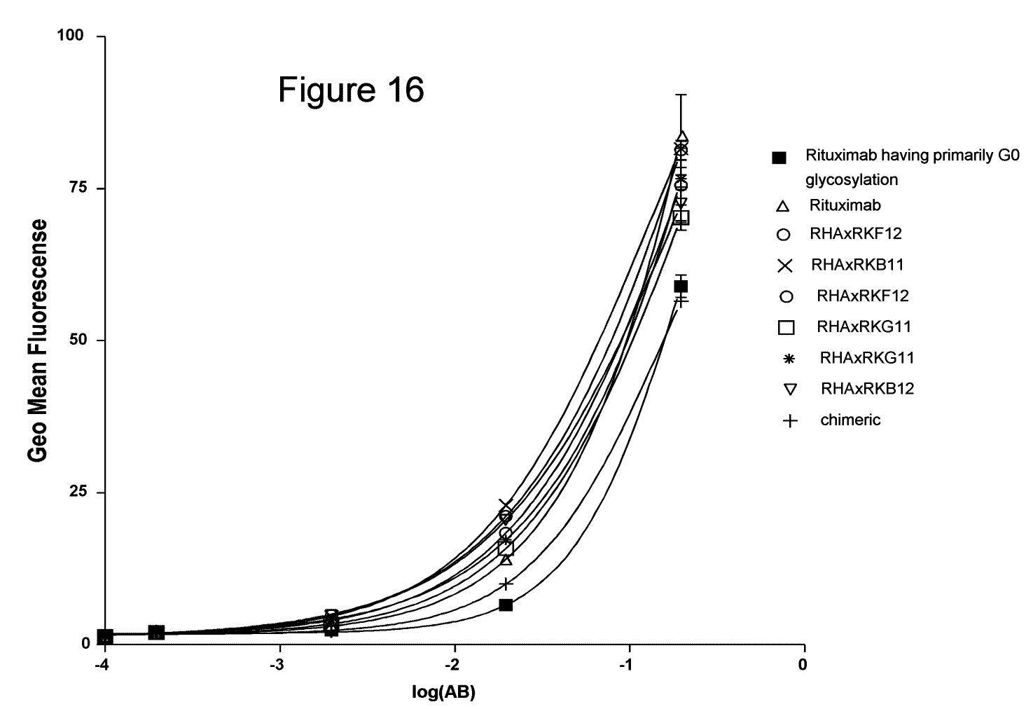

Raji cell binding by the humanized 1F5 antibodies was also analyzed with a FACSort flow cytometer (Becton Dickinson, San Jose Calif.). Standard FACS (fluorescent-activated cell sorting) procedures were utilized for the assays. For measurement of cell binding, debris and cell clumps were gated out based on forward versus side scatter. Dead cells were excluded from analysis (“gated out”) based on PI uptake. 5,000 to 10,000 viable cells were analyzed per sample and the geometric mean of the fluorescence distribution (mean fluorescence intensity, or MFI) was determined using instrument software (CellQuest, Becton-Dicknson). MFI values are reported for all studies. Two studies were conducted for each of humanized antibodies RHA×RKF12; RHA×RKB11; and RHA×RKG11. The results of the FACS binding assay are provided in FIG. 16. As shown in the figure, the humanized antibodies that were tested (RHA×RKF12; RHA×RKB11; and RHA×RKG11) all had higher binding affinity than the chimeric 1F5.

### Example 4

### Thermostability

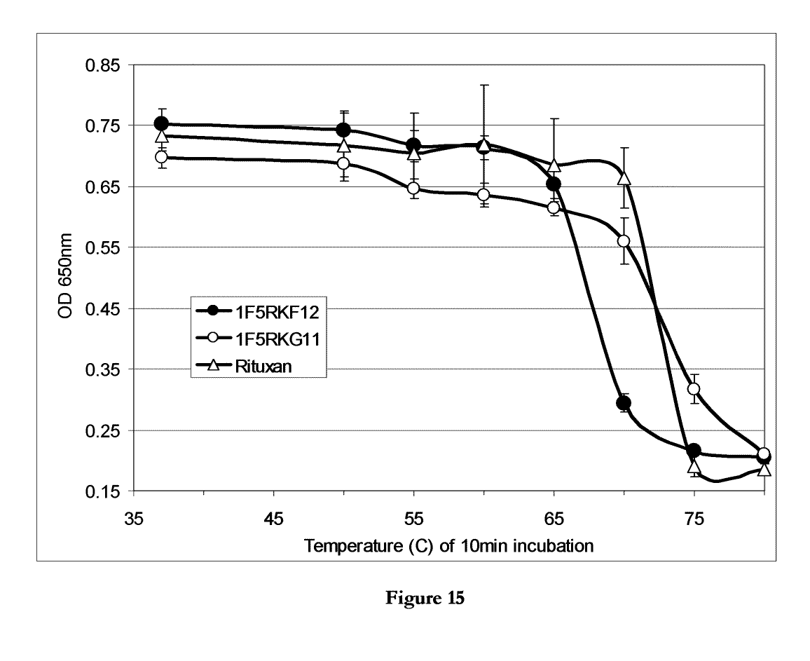

To investigate whether the two antibodies (1F5RKG11×1F5RHA and 1F5RKF12×1F5RHA) were of similar structural stability, 1 μg/ml dilutions of each antibody in culture medium, buffered by PBS, were heated for 10 minutes at seven temperatures between 50 and 80° C. (50°, 55°, 60°, 65°, 70°, 75°, and 80° C.). After rapid cooling to 4° C., Raji binding analysis was performed as described above. The results of the Raji binding assay (FIG. 15) indicated that the 1F5RKG11×1F5RHA antibody retained binding potency at a temperature higher than did the 1F5RKF12×1F5RHA antibody. At 75° C. the 1F5RKG11 antibody retained a greater residual potency than Rituxan, which was inactivated by 10 minutes at this temperature.

### Example 5

### CDC Activity, Off-Rate, and B-Cell Depletion of Humanized 1F5 Antibodies

Four of the humanized 1F5 antibodies described above (RHA×RKB11 (Variant D); RHA×RKG11 (Variant E); RHA×RKB12 (Variant F); and RHA×RKF12 (Variant G)) as well as chimeric antibody c1F5 were produced in transgenic Lemna plants providing for primarily G0 glycosylation. Analysis of the chimeric and humanized antibodies (e.g., by matrix-assisted laser desorption ionization time-of-flight mass spectrometry (MALDI-TOF MS) analysis) confirmed that the antibodies had predominantly G0 glycosylation (data not shown).

The purpose of the present example was to evaluate several aspects of the in vitro activity of these antibodies, including complement-dependent cytotoxic activity (CDC) using Raji cells as targets, dissociation of binding (“off rate”) from B-cells, and in vitro B-cell depleting activity in whole blood. The values for rituximab (RTX) were also measured and compared to the values determined for the humanized and chimeric 1F5 antibodies.

### CDC Activity

Complement dependent cytotoxicity (CDC) of variant antibodies was measured in Raji Burkitt Lymphoma cells (ATCC Accession Number CCL-86) using flow cytometry to enumerate dead cells. Briefly, cells were treated with varying concentrations of different antibodies in 90 microliters PBS followed by the addition of 10 microliters normal human serum to give a final concentration of 10% serum. Cells were incubated for 30 minutes at 37° C. and then placed on ice. Cold (4° C.) PBS containing propidium iodide (PI) was added to the cells and the frequency of PI-positive cells determined by FACS.

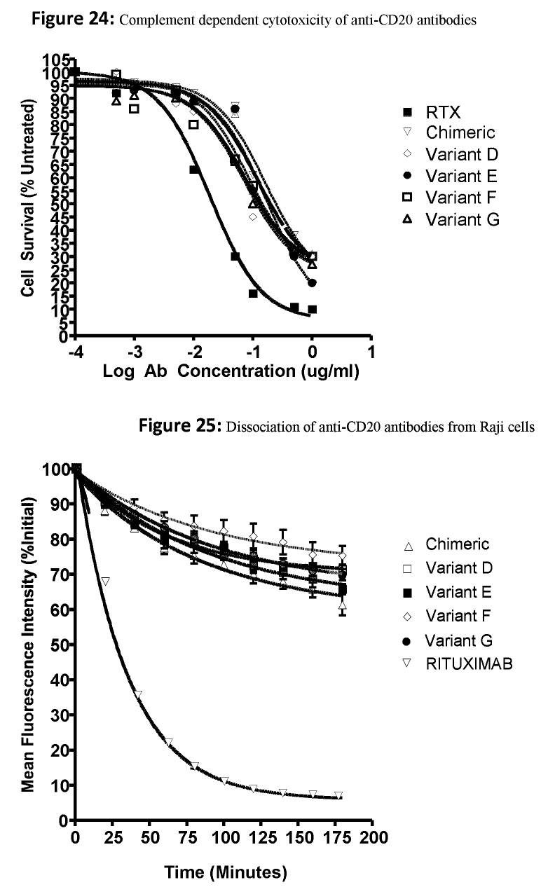

FIG. 24 shows the results of the CDC assay of various concentrations of the tested antibodies. As shown in the Figure, the G0-glycosylated humanized 1F5 antibodies had much less complement dependent cytotoxicity than rituximab, with the CDC of antibody E being approximately 10 times less than rituximab.

In terms of EC₅₀'s, the antibodies ranked as follows:

RTX<Variant G<Variant F<Variant D<chimeric<Variant E (Table 2).

TABLE 2

| Variant | RTX | chimeric | Variant D | Variant E | Variant F | Variant G |
|---|---|---|---|---|---|---|
| LogEC50 | −1.726 | −0.7810 | −1.063 | −0.773 | −1.133 | −1.146 |
| EC50 | 0.01879 | 0.1656 | 0.08642 | 0.1685 | 0.07368 | 0.07138 |

Antibody Off-Rates from Cells

The rate at which fluorescently-labeled antibodies dissociated from Raji cells was compared with RTX over a 4-hour time period. The assay format used followed that described in Goldenberg D. M et al. (2009. Blood, Vol 113(5):1062) and is briefly outlined below.

All antibodies were conjugated to a fluorescein-like fluorophore, DyLight 488, according to vendor instructions (Pierce Cat. No. 53025). Following conjugation, labeled antibody was separated from free dye using chromatography columns provided in the labeling kit. Protein concentration after labeling was measured spectrophotometrically and concentrations found to vary between 0.93 and 1.1 mg/ml. Purified labeled antibodies were stored at 4° C. in PBS protected from light.

To measure antibody dissociation from cells, 10⁶ Raji cells were suspended in PBS containing 1% bovine serum albumin (PBS/BSA). Labeled antibody was added to a final concentration of 5 ug/ml in a volume of 1.0 ml. Following a 30 minute incubation at 37° C., unlabeled (“cold”) antibody was added to a final concentration of 1000 ug/ml. Labeled cells mixed with cold antibody were returned to 37° C. for 4 hours. At 20-minute intervals, 100 ul aliquots of cells were withdrawn from each sample, 400 uL cold PBS/BSA containing propidium iodide was added and mean fluorescence intensity (MFI) of the cell population excluding dead cells (PI⁺) determined by FACS. It was presumed with this protocol that labeling does not influence antibody affinity.

It was observed that the humanized 1F5 antibodies had significantly decreased off-rates relative to rituximab (FIG. 25). As shown in FIG. 25, it is clear that the humanized 1F5 antibodies show roughly a 2-3 fold increase in half-life relative to rituximab.

The results of off-rate studies are summarized in Table 3. Although antibody Variants D and F showed some variation in half-life in the two studies, overall the half-lives were similar. All humanized 1F5 antibodies had significantly decreased off rates as compared to rituximab (i.e., the humanized 1F5 antibodies bound ˜2-3 times longer than rituximab).

TABLE 3
Half-life values for anti-CD20 antibodies

| | | | | | Variant | |
|---|---|---|---|---|---|---|
| | chimeric | Variant D | Variant E | Variant F | G | RTX |
| Study 1 | 59 | 44 | 67 | 72 | 67 | 24 |
| Study 2 | 57 | 59 | 63 | 64 | 65 | 24 |
| Average | 58 | 52 | 65 | 68 | 66 | 24 |

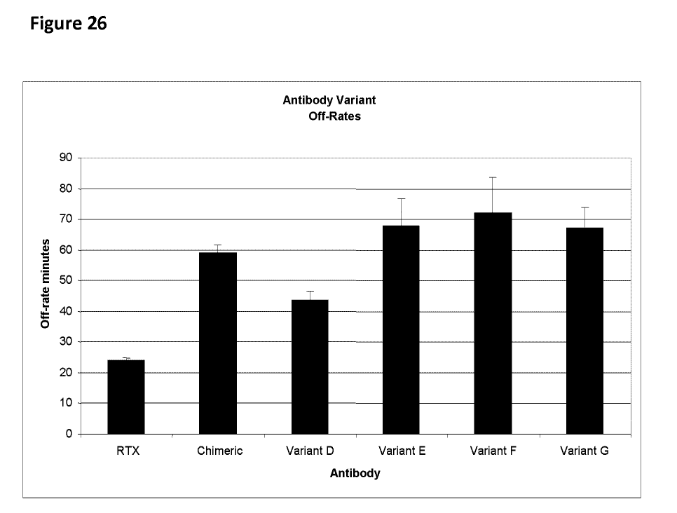

FIG. 26 shows a bar graph of the half-lives obtained from the anti-CD20 antibodies in Study 1.

### B-Cell Depletion in Whole Blood

The B-cell depleting activity of the humanized 1F5 antibodies was evaluated. Briefly, test antibodies were added to 100 ul fresh whole blood at specified concentrations and incubated for 4 hours at 37° C. The frequency of B cells was determined using CD19, a B-cell specific antibody that binds cells independently of CD20 receptor expression. Two studies were performed, one in which CD16 was not blocked with an anti-CD16 antibody prior to exposure to test antibody and one in which CD16 was pre-blocked.

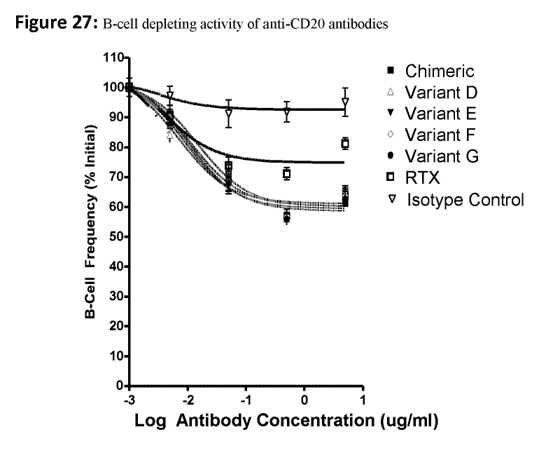

In the first set of experiments without anti-CD16, the cytotoxicity of the humanized 1F5 antibodies was observed to be greater than that of rituximab (FIG. 27). That is, the humanized 1F5 antibodies deplete B cells more effectively than rituximab. FIG. 27 shows the results of the first set of experiments for the chimeric 1F5, variants D-G, rituximab, and an isotype control (i.e., a control IgG1, G0 antibody with no B-cell antigen binding properties).

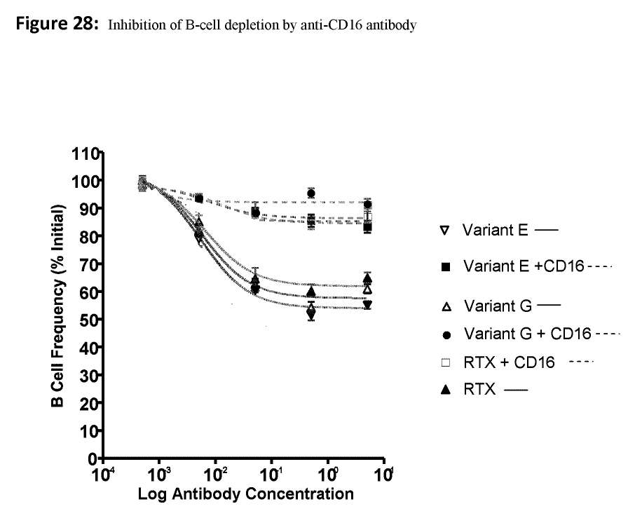

In the second set of experiments, CD16 receptor on effector cells was pre-blocked with anti-CD16 antibody. When humanized antibody variants E and G were then tested in the B-cell depletion assay, it was shown that the B-cell depleting activity of the anti-CD20 antibodies was inhibited by blocking CD16 on effector cells with an anti-CD16 antibody (FIG. 28). The effect of anti-CD16 was to reduce B-cell depleting activity almost completely, implicating ADCC as the major mode of action for the humanized 1F5 antibodies. Anti-CD16 antibody inhibited B-cell depletion for variants E and G similarly to rituximab.

### CONCLUSION

I. The humanized 1F5 antibodies have much lower CDC activity than rituximab.

II. The rate at which all of the humanized 1F5 antibodies dissociated from cells (i.e., the off-rate) was significantly reduced relative to the off-rate of rituximab.

III. All of the humanized 1F5 antibodies were more efficient than rituximab in depleting B-cells from whole blood.

IV. For the two humanized 1F5 antibody variants tested, B-cell depletion in whole blood was almost completely inhibited by treatment with a CD16-blocking antibody.

While the invention has been described in detail and with reference to specific embodiments thereof, it will be apparent to one skilled in the art that various changes and modifications can be made without departing from the spirit and scope of the invention.

What is claimed is:

1. A humanized antibody comprising:
(a) a light chain comprising a light chain variable region comprising an amino acid sequence selected from the group consisting of:
(SEQ ID NO: 1)
(1) Glu Ile Val Leu Thr Gln Ser Pro Ser Ser Leu
Ser Ala Ser Val Gly Asp Arg Val Thr Ile Thr Cys
Arg Ala Ser Ser Ser Leu Ser Phe Met His Trp Tyr
Gln Gln Lys Pro Gly Lys Ala Pro Lys Xaa₄₅ Xaa₄₆
Ile Tyr Ala Thr Ser Asn Leu Ala Ser Gly Val Pro
Ser Arg Phe Ser Gly Ser Gly Ser Gly Thr Asp Xaa₇₀
Thr Leu Thr Ile Ser Ser Leu Gln Pro Glu Asp Phe
Ala Thr Tyr Xaa₈₆ Cys His Gln Trp Ser Ser Asn Pro
Leu Thr Phe Gly Gly Gly Thr Lys Val Glu Ile Lys
wherein
Xaa₄₅ is Pro or Leu;
Xaa₄₆ is Trp or Leu;
Xaa₇₀ is Tyr or Phe; and
Xaa₈₆ is Tyr or Phe; and
(SEQ ID NO: 2)
(2) Glu Ile Val Leu Thr Gln Ser Pro Gly Thr Leu
Ser Leu Ser Pro Gly Glu Arg Ala Thr Leu Ser Cys
Arg Ala Ser Ser Ser Leu Ser Phe Met His Trp Tyr
Gln Gln Lys Pro Gly Gln Ala Pro Arg Xaa₄₅ Xaa₄₆
Ile Tyr Ala Thr Ser Asn Leu Ala Ser Gly Ile Pro
Ala Arg Phe Ser Gly Ser Gly Ser Gly Thr Asp Xaa₇₀
Thr Leu Thr Ile Ser Arg Leu Glu Pro Glu Asp Phe
Ala Val Tyr Phe Cys His Gln Trp Ser Ser Asn Pro
Leu Thr Phe Gly Gln Gly Thr Lys Val Glu Leu Lys
wherein
Xaa₄₅ is Pro or Leu;
Xaa₄₆ is Trp or Leu; and
Xaa₇₀ is Tyr or Phe; and
(b) a heavy chain comprising a heavy chain variable region comprising the following amino acid sequence:
(SEQ ID NO: 3)
Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr Xaa₇₀
Thr Xaa₇₂ Asp Xaa₇₄ Ser Ala Asn Thr Ala Tyr Met
Glu Leu Ser Ser Leu Lys Ser Glu Asp Thr Ala Val
Tyr Tyr Cys Ala Arg Ser His Tyr Gly Ser Asn Tyr
Val Asp Tyr Phe Asp Tyr Trp Gly Gln Gly Thr Leu
Val Thr Val Ser Ser
wherein
Xaa₆₈ is Val or Ala;
Xaa₇₀ is Val or Leu;
Xaa₇₂ is Arg or Ala; and
Xaa₇₄ is Thr or Lys;
wherein the humanized antibody binds to human CD-20.

2. The humanized antibody of claim 1, wherein the heavy chain variable region comprises the amino acid sequence of SEQ ID NO: 13.

3. The humanized antibody of claim 1, wherein the light chain variable region comprises the amino acid sequence of SEQ ID NO: 54.

4. The humanized antibody of claim 1, wherein the light chain variable region comprises the amino acid sequence of SEQ ID NO: 49.

5. The humanized antibody of claim 1, wherein the light chain variable region comprises the amino acid sequence of SEQ ID NO: 59.

6. The humanized antibody of claim 1, wherein the light chain variable region comprises the amino acid sequence of SEQ ID NO: 55.

7. The humanized antibody of claim 1, wherein the heavy chain variable region comprises the amino acid sequence of SEQ ID NO: 13 and the light chain variable region comprises an amino acid sequence selected from the group consisting of SEQ ID NOS: 49, 54, 55, and 59.

8. The humanized antibody of claim 1, wherein the heavy chain variable region comprises an amino acid sequence selected from the group consisting of SEQ ID NOS: 13 and 48.

9. The humanized antibody of claim 1, wherein the light chain variable region comprises an amino acid sequence selected from the group consisting of SEQ ID NOS: 28, 49, 50, 51, 52, 53, 54, 42, 55, 56, 57, 58 and 59.

10. The humanized antibody of claim 8, wherein the light chain variable region comprises an amino acid sequence selected from the group consisting of SEQ ID NOS: 28, 49, 50, 51, 52, 53, 54, 42, 55, 56, 57, 58 and 59.

11. The humanized antibody of claim 1, wherein the antibody binds to human CD20 with an affinity similar to that of chimeric antibody c1F5.

12. The humanized antibody of claim 1, wherein the antibody binds to human CD20 with an affinity greater than that of chimeric antibody c1F5.

13. The humanized antibody of claim 1, wherein the antibody binds to human CD20 with an off-rate lower than that of chimeric antibody c1F5.

14. The humanized antibody of claim 1, wherein the light chain further comprises a human light chain constant region.

15. The humanized antibody of claim 14, wherein the light chain comprises an amino acid sequence selected from the group consisting of SEQ ID NOS: 60, 61, 62, and 63.

16. The humanized antibody of claim 1, wherein the heavy chain further comprises a human heavy chain constant region.

17. The humanized antibody of claim 16, wherein the heavy chain comprises the amino acid sequence of SEQ ID NO: 64.

18. The humanized antibody of claim 14, wherein the heavy chain further comprises a human heavy chain constant region.

19. The humanized antibody of claim 18, wherein the heavy chain comprises the amino acid sequence of SEQ ID NO: 64 and the light chain comprises an amino acid sequence selected from the group consisting of SEQ ID NOS: 60, 61, 62, and 63.

20. The humanized antibody of claim 19, wherein the heavy chain comprises the amino acid sequence of SEQ ID NO: 64 and the light chain comprises the amino acid sequence of SEQ ID NO: 60.

21. The humanized antibody of claim 19, wherein the heavy chain comprises the amino acid sequence of SEQ ID NO: 64 and the light chain comprises the amino acid sequence of SEQ ID NO: 61.

22. The humanized antibody of claim 19, wherein the heavy chain comprises the amino acid sequence of SEQ ID NO: 64 and the light chain comprises the amino acid sequence of SEQ ID NO: 62.

23. The humanized antibody of claim 19, wherein the heavy chain comprises the amino acid sequence of SEQ ID NO: 64 and the light chain comprises the amino acid sequence of SEQ ID NO: 63.

24. A pharmaceutical composition comprising the humanized antibody of any of the preceding claims and a pharmaceutically acceptable excipient.

25. A pharmaceutical composition comprising the humanized antibody of claim 7 and a pharmaceutically acceptable excipient.

26. The humanized antibody of claim 1, wherein the antibody is produced in a host cell selected from the group consisting of a mammalian cell, a yeast cell, and a plant cell.

27. A humanized antibody comprising a light chain comprising a light chain variable region comprising the following amino acid sequence:
(SEQ ID NO: 1)
Glu Ile Val Leu Thr Gln Ser Pro Ser Ser Leu Ser
Ala Ser Val Gly Asp Arg Val Thr Ile Thr Cys Arg
Ala Ser Ser Ser Leu Ser Phe Met His Trp Tyr Gln
Gln Lys Pro Gly Lys Ala Pro Lys Xaa₄₅ Xaa₄₆
Ile Tyr Ala Thr Ser Asn Leu Ala Ser Gly Val Pro
Ser Arg Phe Ser Gly Ser Gly Ser Gly Thr Asp Xaa₇₀
Thr Leu Thr Ile Ser Ser Leu Gln Pro Glu Asp Phe
Ala Thr Tyr Xaa₈₆ Cys His Gln Trp Ser Ser
Asn Pro Leu Thr Phe Gly Gly Gly Thr Lys Val Glu
Ile Lys
wherein
Xaa₄₅ is Pro or Leu;
Xaa₄₆ is Trp or Leu;
Xaa₇₀ is Tyr or Phe; and
Xaa₈₆ is Tyr or Phe;
wherein the humanized antibody binds to human CD-20.

28. The humanized antibody of claim 27, wherein the light chain variable region has the amino acid sequence of SEQ ID NO: 54.

29. The humanized antibody of claim 27, wherein the light chain variable region has the amino acid sequence of SEQ ID NO: 49.

30. The humanized antibody of claim 27, wherein the light chain variable region comprises an amino acid sequence selected from the group consisting of SEQ ID NOS: 28, 49, 50, 51, 52, 53, and 54.

31. The humanized antibody of claim 27, wherein the antibody binds to human CD20 with an affinity similar to that of chimeric antibody c1F5.

32. The humanized antibody of claim 27, wherein the antibody binds to human CD20 with an affinity greater than that of chimeric antibody c1F5.

33. The humanized antibody of claim 27, wherein the antibody binds to human CD20 with an off-rate lower than that of chimeric antibody c1F5.

34. A humanized antibody comprising a light chain comprising a light chain variable region comprising the following amino acid sequence:
(SEQ ID NO: 2)
Glu Ile Val Leu Thr Gln Ser Pro Gly Thr Leu Ser
Leu Ser Pro Gly Glu Arg Ala Thr Leu Ser Cys Arg
Ala Ser Ser Ser Leu Ser Phe Met His Trp Tyr Gln
Gln Lys Pro Gly Gln Ala Pro Arg Xaa₄₅ Xaa₄₆
Ile Tyr Ala Thr Ser Asn Leu Ala Ser Gly Ile Pro
Ala Arg Phe Ser Gly Ser Gly Ser Gly Thr Asp Xaa₇₀
Thr Leu Thr Ile Ser Arg Leu Glu Pro Glu Asp Phe
Ala Val Tyr Phe Cys His Gln Trp Ser Ser Asn Pro
Leu Thr Phe Gly Gln Gly Thr Lys Val Glu Leu Lys
wherein
Xaa₄₅ is Pro or Leu;
Xaa₄₆ is Trp or Leu; and
Xaa₇₀ is Tyr or Phe;
wherein the humanized antibody binds to human CD-20.

35. The humanized antibody of claim 34, wherein the light chain variable region comprises the amino acid sequence of SEQ ID NO: 59.

36. The humanized antibody of claim 34, wherein the light chain variable region comprises the amino acid sequence of SEQ ID NO: 55.

37. The humanized antibody of claim 34, wherein the light chain variable region comprises an amino acid sequence selected from the group consisting of SEQ ID NOS: 42, 55, 56, 57, 58 and 59.

38. The humanized antibody of claim 34, wherein the antibody binds to human CD20 with an affinity similar to that of chimeric antibody c1F5.

39. The humanized antibody of claim 34, wherein the antibody binds to human CD20 with an affinity greater than that of chimeric antibody c1F5.

40. The humanized antibody of claim 34, wherein the antibody binds to human CD20 with an off-rate lower than that of chimeric antibody c1F5.

41. A humanized antibody comprising a heavy chain comprising a heavy chain variable region comprising the following amino acid sequence:
(SEQ ID NO: 3)
Gln Val Gln Leu Val Gln Ser Gly Ala Glu Val Lys
Lys Pro Gly Ala Ser Val Lys Val Ser Cys Lys Ala
Ser Gly Tyr Thr Phe Thr Ser Tyr Asn Met His Trp
Val Arg Gln Ala Pro Gly Gln Arg Leu Glu Trp Ile
Gly Ala Ile Tyr Pro Gly Asn Gly Asp Thr Ser Tyr
Asn Gln Lys Phe Lys Gly Arg Xaa₆₈ Thr Xaa₇₀ Thr
Xaa₇₂ Asp Xaa₇₄ Ser Ala Asn Thr Ala Tyr Met Glu
Leu Ser Ser Leu Lys Ser Glu Asp Thr Ala Val Tyr
Tyr Cys Ala Arg Ser His Tyr Gly Ser Asn Tyr Val
Asp Tyr Phe Asp Tyr Trp Gly Gln Gly Thr Leu Val
Thr Val Ser Ser
wherein
Xaa₆₈ is Val or Ala;
Xaa₇₀ is Val or Leu;
Xaa₇₂ is Arg or Ala; and
Xaa₇₄ is Thr or Lys;
wherein the humanized antibody is capable of binding to human CD-20.

42. The humanized antibody of claim 41, wherein the antibody binds to human CD20 with an affinity similar to that of chimeric antibody c1F5.

43. The humanized antibody of claim 41, wherein the antibody binds to human CD20 with an affinity greater than that of chimeric antibody c1F5.

44. The humanized antibody of claim 41, wherein the antibody binds to human CD20 with an off-rate lower than that of chimeric antibody c1F5.

45. The humanized antibody of claim 41, wherein the heavy chain variable region comprises the amino acid sequence of SEQ ID NO: 13.

46. The humanized antibody of claim 41, wherein the heavy chain variable region comprises the amino acid sequence of SEQ ID NO: 48.

47. A method of treating a B-cell disorder in a subject comprising administering to a subject in need thereof a therapeutically effective amount of the antibody of claim 1, 27, 34, or 41.

48. A method of preventing a B-cell disorder in a subject comprising administering to the subject a prophylactically effective amount of the antibody of claim 1, 27, 34, or 41.

49. A humanized anti-CD20 antibody composition comprising the humanized antibody of claim 1, 27, 34, or 41, wherein at least 90% of the N-glycans present in said composition are GlcNAc₂Man₃GlcNAc₂ (G0).

50. An isolated nucleic acid comprising a nucleic acid sequence encoding the antibody of claim 1, 27, 34, or 41.

51. A host cell comprising the isolated nucleic acid of claim 50.


SEQUENCE LISTING (97 sequences)

SEQ ID NO: 1 - protein, 106 aa, Artificial Sequence
  Description of Artificial Sequence Synthetic polypeptide
EIVLTQSPSSLSASVGDRVTITCRASSSLSFMHWYQQKPGKAPKXXIYATSNLASGVPSRFSGSGSGTDXTLTISSLQPEDFATYXCHQWSSNPLTFGGGTKVEIK

SEQ ID NO: 2 - protein, 106 aa, Artificial Sequence
  Description of Artificial Sequence Synthetic polypeptide
EIVLTQSPGTLSLSPGERATLSCRASSSLSFMHWYQQKPGQAPRXXIYATSNLASGIPARFSGSGSGTDXTLTISRLEPEDFAVYFCHQWSSNPLTFGQGTKVELK

SEQ ID NO: 3 - protein, 122 aa, Artificial Sequence
  Description of Artificial Sequence Synthetic polypeptide
QVQLVQSGAEVKKPGASVKVSCKASGYTFTSYNMHWVRQAPGQRLEWIGAIYPGNGDTSYNQKFKGRXTXTXDXSANTAYMELSSLKSEDTAVYYCARSHYGSNYVDYFDYWGQGTLVTVSS

SEQ ID NO: 4 - DNA, 57 bp, Homo sapiens
atggactggacctggaggatcctctttttggtggcagcagccacaggtgcccactcc

SEQ ID NO: 5 - DNA, 90 bp, Homo sapiens
caggtccagcttgtgcagtctggggctgaggtgaagaagcctggggcctcagtgaaggtttcctgcaaggcttctggatacaccttcact

SEQ ID NO: 6 - DNA, 15 bp, Artificial Sequence
  Description of Artificial Sequence Synthetic oligonucleotide
agttacaatatgcac

SEQ ID NO: 7 - DNA, 42 bp, Homo sapiens
tgggtgcgccaggcccccggacaaaggcttgagtggatagga

SEQ ID NO: 8 - DNA, 51 bp, Artificial Sequence
  Description of Artificial Sequence Synthetic oligonucleotide
gctatttatccaggaaatggtgatacttcctacaatcagaagttcaaaggc

SEQ ID NO: 9 - DNA, 96 bp, Homo sapiens
agagtcaccgttaccagggacacatccgcgaacacagcctacatggagctgagcagcctaaaatctgaagacacggctgtgtattactgtgcgaga

SEQ ID NO: 10 - DNA, 39 bp, Artificial Sequence
  Description of Artificial Sequence Synthetic oligonucleotide
tcgcactacggtagtaactacgtagactactttgactac

SEQ ID NO: 11 - DNA, 33 bp, Homo sapiens
tggggccagggaaccctggtcaccgtctcctca

SEQ ID NO: 12 - protein, 122 aa, Artificial Sequence
  Description of Artificial Sequence Synthetic polypeptide
QVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTLVTVST

SEQ ID NO: 13 - protein, 122 aa, Artificial Sequence
  Description of Artificial Sequence Synthetic polypeptide
QVQLVQSGAEVKKPGASVKVSCKASGYTFTSYNMHWVRQAPGQRLEWIGAIYPGNGDTSYNQKFKGRVTVTRDTSANTAYMELSSLKSEDTAVYYCARSHYGSNYVDYFDYWGQGTLVTVSS

SEQ ID NO: 14 - protein, 121 aa, Homo sapiens
QVQLVQSGAEVKKPGASVKVSCKASGYTFTNYAIHWVRQAPGQRLEWIGWINTASANTKCSQNFQGRVTVTRDTSANTAYMELSSLKSEDTAVYYCARCSGDTCYQGSDYWGQGTLVTVSS

SEQ ID NO: 15 - DNA, 456 bp, Artificial Sequence
  Description of Artificial Sequence Synthetic polynucleotide
aagcttgccgccaccatggactggacctggcggatcctgttcctggtggctgctgccacaggcgctcactcccaggtccagctggtgcagtctggcgccgaggtcaagaagcctggcgcctccgtcaaggtgtcctgcaaggcctccggctacaccttcacctcctacaacatgcactgggtgcggcaggcccctggccagcggctggaatggatcggcgccatctaccctggcaacggcgacaccagctacaaccagaagttcaagggcagagtcacagtgacccgggacacctccgccaacaccgcctacatggaactgtcctccctgaagtccgaggacaccgccgtgtactactgcgcccggtcccactacggctccaactacgtggactacttcgactactggggccagggcaccctggtgaccgtgtcctccgcctccaccaagggcccn

SEQ ID NO: 16 - protein, 152 aa, Artificial Sequence
  Synthetic Construct
KLAATMDWTWRILFLVAAATGAHSQVQLVQSGAEVKKPGASVKVSCKASGYTFTSYNMHWVRQAPGQRLEWIGAIYPGNGDTSYNQKFKGRVTVTRDTSANTAYMELSSLKSEDTAVYYCARSHYGSNYVDYFDYWGQGTLVTVSSASTKGP

SEQ ID NO: 17 - DNA, 456 bp, Artificial Sequence
  Description of Artificial Sequence Synthetic polynucleotide
aagcttgccgccaccatggactggacctggcggatcctgttcctggtggctgctgccacaggcgctcactcccaggtccagctggtgcagtctggcgccgaggtcaagaagcctggcgcctccgtcaaggtgtcctgcaaggcctccggctacaccttcacctcctacaacatgcactgggtgcggcaggcccctggccagcggctggaatggatcggcgccatctaccctggcaacggcgacaccagctacaaccagaagttcaagggcagagccacactgaccgccgacaagtccgccaacaccgcctacatggaactgtcctccctgaagtccgaggacaccgccgtgtactactgcgcccggtcccactacggctccaactacgtggactacttcgactactggggccagggcaccctggtgaccgtgtcctccgcctccaccaagggcccn

SEQ ID NO: 18 - protein, 152 aa, Artificial Sequence
  Synthetic Construct
KLAATMDWTWRILFLVAAATGAHSQVQLVQSGAEVKKPGASVKVSCKASGYTFTSYNMHWVRQAPGQRLEWIGAIYPGNGDTSYNQKFKGRATLTADKSANTAYMELSSLKSEDTAVYYCARSHYGSNYVDYFDYWGQGTLVTVSSASTKGP

SEQ ID NO: 19 - DNA, 66 bp, Homo sapiens
atggacatgagggtccccgctcagctcctggggctcctgctgctctggctcccaggtgccagatgt

SEQ ID NO: 20 - DNA, 69 bp, Homo sapiens
gaaattgtgctgactcagtctccatcctccctgtctgcatctgtaggagacagagtcaccatcacttgc

SEQ ID NO: 21 - DNA, 30 bp, Artificial Sequence
  Description of Artificial Sequence Synthetic oligonucleotide
agggccagctcaagtttaagtttcatgcac

SEQ ID NO: 22 - DNA, 45 bp, Homo sapiens
tggtatcagcaaaaaccagggaaagcccctaagctcctgatctat

SEQ ID NO: 23 - DNA, 21 bp, Artificial Sequence
  Description of Artificial Sequence Synthetic oligonucleotide
gccacatccaacctggcttct

SEQ ID NO: 24 - DNA, 96 bp, Homo sapiens
ggggtcccatcaaggttcagcggcagtggatctgggacagatttcactctcaccatcagcagcctgcagcctgaagattttgcaacttattactgt

SEQ ID NO: 25 - DNA, 27 bp, Artificial Sequence
  Description of Artificial Sequence Synthetic oligonucleotide
catcagtggagtagtaacccgctcacg

SEQ ID NO: 26 - DNA, 30 bp, Homo sapiens
ttcggcggagggaccaaggtggagatcaaa

SEQ ID NO: 27 - protein, 106 aa, Artificial Sequence
  Description of Artificial Sequence Synthetic polypeptide
QIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKVEIK

SEQ ID NO: 28 - protein, 106 aa, Artificial Sequence
  Description of Artificial Sequence Synthetic polypeptide
EIVLTQSPSSLSASVGDRVTITCRASSSLSFMHWYQQKPGKAPKLLIYATSNLASGVPSRFSGSGSGTDFTLTISSLQPEDFATYYCHQWSSNPLTFGGGTKVEIK

SEQ ID NO: 29 - protein, 107 aa, Homo sapiens
EIVLTQSPSSLSASVGDRVTITCRASQGISSYLAWYQQKPGKAPKLLIYAASTLQSGVPSRFSGSGSGTDFTLTISSLQPEDFATYYCQQLNSYPLTFGGGTKVEIK

SEQ ID NO: 30 - DNA, 412 bp, Artificial Sequence
  Description of Artificial Sequence Synthetic polynucleotide
aagcttgccgccaccatggacatgcgggtgcccgcacagctgctgggcctgctgctgctgtggctgccaggcgctagatgcgagatcgtgctgacccagtccccttcctccctgtccgcctccgtgggcgaccgggtgaccatcacctgccgggcctcctcctccctgagcttcatgcactggtatcagcagaagcctggcaaggcccctaagctgctgatctacgccacctccaacctggcctccggcgtgccttcccggttctccggctccggcagcggcaccgacttcaccctgaccatctcctccctgcagcctgaggacttcgccacctactactgccaccagtggtcctccaaccctctgacctttggcggcggaacaaaggtggagatcaaacgtgagtggatcc

SEQ ID NO: 31 - protein, 137 aa, Artificial Sequence
  Synthetic Construct
KLAATMDMRVPAQLLGLLLLWLPGARCEIVLTQSPSSLSASVGDRVTITCRASSSLSFMHWYQQKPGKAPKLLIYATSNLASGVPSRFSGSGSGTDFTLTISSLQPEDFATYYCHQWSSNPLTFGGGTKVEIKREWI

SEQ ID NO: 32 - DNA, 412 bp, Artificial Sequence
  Description of Artificial Sequence Synthetic polynucleotide
aagcttgccgccaccatggacatgcgggtgcccgcacagctgctgggcctgctgctgctgtggctgccaggcgccagatgcgagatcgtgctgacccagtccccttcctccctgtccgcctccgtgggcgaccgggtgaccatcacctgccgggcctcctcctccctgagcttcatgcactggtatcagcagaagcctggcaaggcccctaagccttggatctacgccacctccaacctggcctccggcgtgccttcccggttctccggctccggcagcggcaccgactacaccctgaccatctcctccctgcagcctgaggacttcgccacctacttttgccaccagtggtcctccaaccctctgacctttggcggcggaacaaaggtggagatcaaacgtgagtggatcc

SEQ ID NO: 33 - protein, 137 aa, Artificial Sequence
  Synthetic Construct
KLAATMDMRVPAQLLGLLLLWLPGARCEIVLTQSPSSLSASVGDRVTITCRASSSLSFMHWYQQKPGKAPKPWIYATSNLASGVPSRFSGSGSGTDYTLTISSLQPEDFATYFCHQWSSNPLTFGGGTKVEIKREWI

SEQ ID NO: 34 - DNA, 60 bp, Homo sapiens
atggaaaccccagcgcagcttctcttcctcctgctactctggctcccagataccaccgga

SEQ ID NO: 35 - DNA, 69 bp, Homo sapiens
gaaattgtgttgacgcagtctccaggcaccctgtctttgtctccaggggaaagagccaccctctcctgc

SEQ ID NO: 36 - DNA, 30 bp, Artificial Sequence; identical to SEQ ID NO: 21
  Description of Artificial Sequence Synthetic oligonucleotide

SEQ ID NO: 37 - DNA, 45 bp, Homo sapiens
tggtaccagcagaaacctggccaggctcccaggctcctcatctat

SEQ ID NO: 38 - DNA, 21 bp, Artificial Sequence
  Description of Artificial Sequence Synthetic oligonucleotide
gccacatccaacctggcttct

SEQ ID NO: 39 - DNA, 96 bp, Homo sapiens
ggcatcccagccaggttcagtggcagtgggtctgggacagacttcactctcaccatcagcagactggagcctgaagattttgcagtgtatttctgc

SEQ ID NO: 40 - DNA, 27 bp, Artificial Sequence
  Description of Artificial Sequence Synthetic oligonucleotide
catcagtggagtagtaacccgctcacg

SEQ ID NO: 41 - DNA, 30 bp, Homo sapiens
ttcggccaagggaccaaggtggaactcaaa

SEQ ID NO: 42 - protein, 106 aa, Artificial Sequence
  Description of Artificial Sequence Synthetic polypeptide
EIVLTQSPGTLSLSPGERATLSCRASSSLSFMHWYQQKPGQAPRLLIYATSNLASGIPARFSGSGSGTDFTLTISRLEPEDFAVYFCHQWSSNPLTFGQGTKVELK

SEQ ID NO: 43 - protein, 108 aa, Homo sapiens
EIVLTQSPGTLSLSPGERATLSCRASHSVSRAYLAWYQQKPGQAPRLLIYGTSSRATGIPARFSGSGSGTDFTLTISRLEPEDFAVYFCSQAFRVPYTFGQGTKVELK

SEQ ID NO: 44 - DNA, 406 bp, Artificial Sequence
  Description of Artificial Sequence Synthetic polynucleotide
aagcttgccgccaccatggaaacacctgcccagctgctgttcctgctgctgctgtggctgcctgacaccaccggcgagatcgtgctgacccagtcccctggcaccctgtccctgagccctggcgagagagccaccctgtcctgcagagcctcctcctccctgtccttcatgcactggtatcagcagaagccaggacaggcccctcggctgctgatctacgccacctccaacctggcctccggcatccctgcccggttctccggctccggcagcggcaccgacttcaccctgaccatctcccggctggaacctgaggacttcgccgtgtacttctgccaccagtggtcctccaaccctctgaccttcggccagggcaccaaggtggagctgaaacgtgagtggatcc

SEQ ID NO: 45 - protein, 135 aa, Artificial Sequence
  Synthetic Construct
KLAATMETPAQLLFLLLLWLPDTTGEIVLTQSPGTLSLSPGERATLSCRASSSLSFMHWYQQKPGQAPRLLIYATSNLASGIPARFSGSGSGTDFTLTISRLEPEDFAVYFCHQWSSNPLTFGQGTKVELKREWI

SEQ ID NO: 46 - DNA, 406 bp, Artificial Sequence
  Description of Artificial Sequence Synthetic polynucleotide
aagcttgccgccaccatggaaacacctgcccagctgctgttcctgctgctgctgtggctgcctgacaccaccggcgagatcgtgctgacccagtcccctggcaccctgtccctgagccctggcgagagagccaccctgtcctgcagagcctcctcctccctgtccttcatgcactggtatcagcagaagccaggacaggcccctcggccttggatctacgccacctccaacctggcctccggcatccctgcccggttctccggctccggcagcggcaccgactacaccctgaccatctcccggctggaacctgaggacttcgccgtgtacttctgccaccagtggtcctccaaccctctgaccttcggccagggcaccaaggtggagctgaaacgtgagtggatcc

SEQ ID NO: 47 - protein, 135 aa, Artificial Sequence
  Synthetic Construct
KLAATMETPAQLLFLLLLWLPDTTGEIVLTQSPGTLSLSPGERATLSCRASSSLSFMHWYQQKPGQAPRPWIYATSNLASGIPARFSGSGSGTDYTLTISRLEPEDFAVYFCHQWSSNPLTFGQGTKVELKREWI

SEQ ID NO: 48 - protein, 122 aa, Artificial Sequence
  Description of Artificial Sequence Synthetic polypeptide
QVQLVQSGAEVKKPGASVKVSCKASGYTFTSYNMHWVRQAPGQRLEWIGAIYPGNGDTSYNQKFKGRATLTADKSANTAYMELSSLKSEDTAVYYCARSHYGSNYVDYFDYWGQGTLVTVSS

SEQ ID NO: 49 - protein, 106 aa, Artificial Sequence
  Description of Artificial Sequence Synthetic polypeptide
EIVLTQSPSSLSASVGDRVTITCRASSSLSFMHWYQQKPGKAPKPWIYATSNLASGVPSRFSGSGSGTDYTLTISSLQPEDFATYFCHQWSSNPLTFGGGTKVEIK

SEQ ID NO: 50 - protein, 106 aa, Artificial Sequence
  Description of Artificial Sequence Synthetic polypeptide
EIVLTQSPSSLSASVGDRVTITCRASSSLSFMHWYQQKPGKAPKLWIYATSNLASGVPSRFSGSGSGTDYTLTISSLQPEDFATYFCHQWSSNPLTFGGGTKVEIK

SEQ ID NO: 51 - protein, 106 aa, Artificial Sequence
  Description of Artificial Sequence Synthetic polypeptide
EIVLTQSPSSLSASVGDRVTITCRASSSLSFMHWYQQKPGKAPKPLIYATSNLASGVPSRFSGSGSGTDYTLTISSLQPEDFATYFCHQWSSNPLTFGGGTKVEIK

SEQ ID NO: 52 - protein, 106 aa, Artificial Sequence
  Description of Artificial Sequence Synthetic polypeptide
EIVLTQSPSSLSASVGDRVTITCRASSSLSFMHWYQQKPGKAPKPWIYATSNLASGVPSRFSGSGSGTDFTLTISSLQPEDFATYFCHQWSSNPLTFGGGTKVEIK

SEQ ID NO: 53 - protein, 106 aa, Artificial Sequence
  Description of Artificial Sequence Synthetic polypeptide
EIVLTQSPSSLSASVGDRVTITCRASSSLSFMHWYQQKPGKAPKPWIYATSNLASGVPSRFSGSGSGTDYTLTISSLQPEDFATYYCHQWSSNPLTFGGGTKVEIK

SEQ ID NO: 54 - protein, 106 aa, Artificial Sequence
  Description of Artificial Sequence Synthetic polypeptide
EIVLTQSPSSLSASVGDRVTITCRASSSLSFMHWYQQKPGKAPKPLIYATSNLASGVPSRFSGSGSGTDFTLTISSLQPEDFATYYCHQWSSNPLTFGGGTKVEIK

SEQ ID NO: 55 - protein, 106 aa, Artificial Sequence
  Description of Artificial Sequence Synthetic polypeptide
EIVLTQSPGTLSLSPGERATLSCRASSSLSFMHWYQQKPGQAPRPWIYATSNLASGIPARFSGSGSGTDYTLTISRLEPEDFAVYFCHQWSSNPLTFGQGTKVELK

SEQ ID NO: 56 - protein, 106 aa, Artificial Sequence
  Description of Artificial Sequence Synthetic polypeptide
EIVLTQSPGTLSLSPGERATLSCRASSSLSFMHWYQQKPGQAPRLWIYATSNLASGIPARFSGSGSGTDYTLTISRLEPEDFAVYFCHQWSSNPLTFGQGTKVELK

SEQ ID NO: 57 - protein, 106 aa, Artificial Sequence
  Description of Artificial Sequence Synthetic polypeptide
EIVLTQSPGTLSLSPGERATLSCRASSSLSFMHWYQQKPGQAPRPLIYATSNLASGIPARFSGSGSGTDYTLTISRLEPEDFAVYFCHQWSSNPLTFGQGTKVELK

SEQ ID NO: 58 - protein, 106 aa, Artificial Sequence
  Description of Artificial Sequence Synthetic polypeptide
EIVLTQSPGTLSLSPGERATLSCRASSSLSFMHWYQQKPGQAPRPWIYATSNLASGIPARFSGSGSGTDFTLTISRLEPEDFAVYFCHQWSSNPLTFGQGTKVELK

SEQ ID NO: 59 - protein, 106 aa, Artificial Sequence
  Description of Artificial Sequence Synthetic polypeptide
EIVLTQSPGTLSLSPGERATLSCRASSSLSFMHWYQQKPGQAPRPLIYATSNLASGIPARFSGSGSGTDFTLTISRLEPEDFAVYFCHQWSSNPLTFGQGTKVELK

SEQ ID NO: 60 - protein, 213 aa, Artificial Sequence
  Description of Artificial Sequence Synthetic polypeptide
EIVLTQSPSSLSASVGDRVTITCRASSSLSFMHWYQQKPGKAPKPLIYATSNLASGVPSRFSGSGSGTDFTLTISSLQPEDFATYYCHQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 213 aa, Artificial Sequence
  Description of Artificial Sequence Synthetic polypeptide
EIVLTQSPSSLSASVGDRVTITCRASSSLSFMHWYQQKPGKAPKPWIYATSNLASGVPSRFSGSGSGTDYTLTISSLQPEDFATYFCHQWSSNPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 213 aa, Artificial Sequence
  Description of Artificial Sequence Synthetic polypeptide
EIVLTQSPGTLSLSPGERATLSCRASSSLSFMHWYQQKPGQAPRPLIYATSNLASGIPARFSGSGSGTDFTLTISRLEPEDFAVYFCHQWSSNPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 213 aa, Artificial Sequence
  Description of Artificial Sequence Synthetic polypeptide
EIVLTQSPGTLSLSPGERATLSCRASSSLSFMHWYQQKPGQAPRPWIYATSNLASGIPARFSGSGSGTDYTLTISRLEPEDFAVYFCHQWSSNPLTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 452 aa, Artificial Sequence
  Description of Artificial Sequence Synthetic polypeptide
QVQLVQSGAEVKKPGASVKVSCKASGYTFTSYNMHWVRQAPGQRLEWIGAIYPGNGDTSYNQKFKGRVTVTRDTSANTAYMELSSLKSEDTAVYYCARSHYGSNYVDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 418 bp, Artificial Sequence
  Description of Artificial Sequence Synthetic polynucleotide
aagcttgccgccaccatgggcttcaagatggagtcacagttccaggtctttgtattcgtgtttctctggttgtctggtgttgacggacaaattgttctctcccagtctccagcaatcctttctgcatctccaggggagaaggtcacaatgacttgcagggccagctcaagtttaagtttcatgcactggtaccagcagaagccaggatcctcccccaaaccctggatttatgccacatccaacctggcttctggagtccctgctcgcttcagtggcagtgggtctgggacctcttactctctcacaatcagcagagtggaggctgaagatgctgccacttatttctgccatcagtggagtagtaacccgctcacgttcggtgctgggacaaaggtggaaataaaacgtgagtggatcc

SEQ ID NO: 66 - protein, 139 aa, Artificial Sequence
  Synthetic Construct
KLAATMGFKMESQFQVFVFVFLWLSGVDGQIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKVEIKREWI

SEQ ID NO: 67 - DNA, 418 bp, Artificial Sequence
  Description of Artificial Sequence Synthetic polynucleotide
aagcttgccgccaccatgggcttcaagatggaatcccagttccaggtgttcgtgtttgtgttcctgtggctgtccggcgtggacggccagatcgtgctgtcccagtcccctgccatcctgtccgccagccctggcgagaaagtgaccatgacctgccgggcctcctcctccctgtccttcatgcactggtatcagcagaagcctggctcctcccctaagccttggatctacgccacctccaacctggcctccggcgtgcctgcccggttctccggctccggcagcggcacctcctactccctgaccatctcccgggtggaggccgaggacgccgccacctacttttgccaccagtggtcctccaaccctctgaccttcggcgctggcaccaaggtggagatcaaacgtgagtggatcc

SEQ ID NO: 68 - protein, 139 aa, Artificial Sequence; identical to SEQ ID NO: 66
  Synthetic Construct

SEQ ID NO: 69 - DNA, 459 bp, Artificial Sequence
  Description of Artificial Sequence Synthetic polynucleotide
aagcttgccgccaccatggaatggagcgggatctttctctttttggtagcaacagctacagatgtccactcccaggtgcaactgcggcagcctggggctgagctggtgaagcctggggcctcagtgaagatgtcctgcaaggcttctggctacacatttaccagttacaatatgcactgggtaaagcagacacctggacagggcctggaatggattggagctatttatccaggaaatggtgatacttcctacaatcagaagttcaaaggcaaggccacattgactgcagacaaatcctccagcacagcctacatgcagctcagcagtctgacatctgaggactctgcggtctattactgtgcaagatcgcactacggtagtaactacgtagactactttgactactggggccaaggcacactagtcacagtctcgacagcctccaccaagggcccatcg

SEQ ID NO: 70 - protein, 153 aa, Artificial Sequence
  Synthetic Construct
KLAATMEWSGIFLFLVATATDVHSQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTLVTVSTASTKGPS

SEQ ID NO: 71 - DNA, 456 bp, Artificial Sequence
  Description of Artificial Sequence Synthetic polynucleotide
aagcttgccgccaccatggagtggtccggcatcttcctgtttctggtggccaccgccaccgacgtgcactcccaggtgcagctgagacagcctggcgccgagctggtgaagcctggcgcttccgtgaagatgtcctgcaaggcctccggctacaccttcacctcctacaacatgcactgggtgaagcagacccccggccagggcctggagtggatcggcgccatctaccctggcaacggcgacaccagctacaaccagaagttcaagggcaaggccaccctgaccgccgacaagtcctcctccaccgcctacatgcagctgtcctccctgacctccgaggactccgccgtgtactactgcgcccggtcccactacggctccaactacgtggactacttcgactactggggccagggcaccctggtgaccgtgtccaccgcctccaccaagggcccn

SEQ ID NO: 72 - protein, 152 aa, Artificial Sequence
  Synthetic Construct
KLAATMEWSGIFLFLVATATDVHSQVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTLVTVSTASTKGP

SEQ ID NO: 73 - protein, 452 aa, Artificial Sequence
  Description of Artificial Sequence Synthetic polypeptide
QVQLRQPGAELVKPGASVKMSCKASGYTFTSYNMHWVKQTPGQGLEWIGAIYPGNGDTSYNQKFKGKATLTADKSSSTAYMQLSSLTSEDSAVYYCARSHYGSNYVDYFDYWGQGTLVTVSTASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 213 aa, Artificial Sequence
  Description of Artificial Sequence Synthetic polypeptide
QIVLSQSPAILSASPGEKVTMTCRASSSLSFMHWYQQKPGSSPKPWIYATSNLASGVPARFSGSGSGTSYSLTISRVEAEDAATYFCHQWSSNPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 20 bp, Artificial Sequence
  Description of Artificial Sequence Synthetic primer
tgttcctttccatgggtctt

SEQ ID NO: 76 - DNA, 18 bp, Artificial Sequence
  Description of Artificial Sequence Synthetic primer
gtgtgcacgccgctggtc

SEQ ID NO: 77 - protein, 5 aa, Artificial Sequence
  Description of Artificial Sequence Synthetic peptide
SYNMH

SEQ ID NO: 78 - protein, 17 aa, Artificial Sequence
  Description of Artificial Sequence Synthetic peptide
AIYPGNGDTSYNQKFKG

SEQ ID NO: 79 - protein, 13 aa, Artificial Sequence
  Description of Artificial Sequence Synthetic peptide
SHYGSNYVDYFDY

SEQ ID NO: 80 - protein, 30 aa, Artificial Sequence
  Description of Artificial Sequence Synthetic peptide
QVQLVQSGAEVKKPGASVKVSCKASGYTFT

SEQ ID NO: 81 - protein, 14 aa, Artificial Sequence
  Description of Artificial Sequence Synthetic peptide
WVRQAPGQRLEWIG

SEQ ID NO: 82 - protein, 32 aa, Artificial Sequence
  Description of Artificial Sequence Synthetic peptide
RVTVTRDTSANTAYMELSSLKSEDTAVYYCAR

SEQ ID NO: 83 - protein, 11 aa, Artificial Sequence
  Description of Artificial Sequence Synthetic peptide
WGQGTLVTVSS

SEQ ID NO: 84 - protein, 10 aa, Artificial Sequence
  Description of Artificial Sequence Synthetic peptide
RASSSLSFMH

SEQ ID NO: 85 - protein, 7 aa, Artificial Sequence
  Description of Artificial Sequence Synthetic peptide
ATSNLAS

SEQ ID NO: 86 - protein, 9 aa, Artificial Sequence
  Description of Artificial Sequence Synthetic peptide
HQWSSNPLT

SEQ ID NO: 87 - protein, 23 aa, Artificial Sequence
  Description of Artificial Sequence Synthetic peptide
EIVLTQSPSSLSASVGDRVTITC

SEQ ID NO: 88 - protein, 15 aa, Artificial Sequence
  Description of Artificial Sequence Synthetic peptide
WYQQKPGKAPKLLIY

SEQ ID NO: 89 - protein, 32 aa, Artificial Sequence
  Description of Artificial Sequence Synthetic polypeptide
GVPSRFSGSGSGTDFTLTISSLQPEDFATYYC

SEQ ID NO: 90 - protein, 10 aa, Artificial Sequence
  Description of Artificial Sequence Synthetic peptide
FGGGTKVEIK

SEQ ID NO: 91 - protein, 10 aa, Artificial Sequence
  Description of Artificial Sequence Synthetic peptide
RASSSLSFMH

SEQ ID NO: 92 - protein, 7 aa, Artificial Sequence
  Description of Artificial Sequence Synthetic peptide
ATSNLAS

SEQ ID NO: 93 - protein, 9 aa, Artificial Sequence
  Description of Artificial Sequence Synthetic peptide
HQWSSNPLT

SEQ ID NO: 94 - protein, 23 aa, Artificial Sequence
  Description of Artificial Sequence Synthetic peptide
EIVLTQSPGTLSLSPGERATLSC

SEQ ID NO: 95 - protein, 15 aa, Artificial Sequence
  Description of Artificial Sequence Synthetic peptide
WYQQKPGQAPRLLIY

SEQ ID NO: 96 - protein, 32 aa, Artificial Sequence
  Description of Artificial Sequence Synthetic polypeptide
GIPARFSGSGSGTDFTLTISRLEPEDFAVYFC

SEQ ID NO: 97 - protein, 10 aa, Artificial Sequence
  Description of Artificial Sequence Synthetic peptide
FGQGTKVELK